(19) United States
(12) Patent Application Publication
(10) Pub. No.: US 2004/0132109 A1
(43) Pub. Date: Jul. 8, 2004

(54) Method

(76) Inventors: Masato Enari, Chuo-ku (JP); Eckhard Flechsig, Versbacher (DE); John Collinge, Queen (GB); Charles Weismann, London (GB)

Correspondence Address: MARSHALL, GERSTEIN & BORUN LLP, 6300 SEARS TOWER, 233 S. WACKER DRIVE, CHICAGO, IL, 60606, US

(21) Appl. No.: 10/470,022

(22) PCT Filed: Jan. 22, 2002
(86) PCT No.: PCT/GB02/00257

(30) Foreign Application Priority Data
Jan. 23, 2001 (GB) 0101762.3

(51) Int. Cl. G01N 33/53; G01N 33/567; C12M 1/34

(52) U.S. Cl. 435/7.2; 435/287.2

(57) ABSTRACT

The invention relates to methods for determining the presence of prions in a tissue/organ or fluid therefrom; said method comprising the steps of: contacting the tissue/organ with one or more devices, wherein said devices are capable of binding prions; removing said devices from contact with said tissue/organ; determining if said devices are binding prions wherein the device is contacted with the tissue/organ for 120 minutes.

### FIELD OF INVENTION

The present invention relates to a method. In artic resent invention relates to an assay method for detecting the presence of prion protein.

### BACKGROUND ART

By way of background information, a prion is a transmissible particle devoid of nucleic acid. The prion protein (PrP) gene encodes prion proteins. The normal form of PrP is called PrPc; the abnormal conformational isomer is called PrPSc and is believed to be the main or only component of the prion. The most notable prion diseases are Bovine Spongiform Encephalopathy (BSE), Scrapie of Sheep and Creutzfeldt-Jakob Disease (CJD) of humans. The most common manifestation of CJD is sporadic CJD (sCJD), which occurs spontaneously in individuals. Iatrogenic CJD (iCJD) is a disease that results from accidental infection. Familial CJD (fCJD) is a form of CJD that occurs in rare families and is caused by mutations of the human PrP gene. Gerstmann-Strassler-Scheinker Disease (GSS) is also a rare inherited form of human prion disease. Both familial diseases are autosomal dominant disorders. ‘New variant’ CJD (vCJD) of humans is a distinct strain type of CJD that is associated with a pattern of PrP glycoforms that are different from those found for other types of CJD. It has been suggested that BSE may have passed from cattle resulting in vCJD in humans.

Prions are unusually resistant to physical and chemical inactivation, which causes problems when sterilising prion-containing material by conventional methods such as heat sterilisation and formaldehyde (Taylor et al. (1994), Arch. Virol. 139, 313-326; Brown et al. (1982), N. Engl. J. Med. 306, 1279-1282; Ernst & Race (1993), J. Virol. Methods 41, 193-201; Taylor (1993), Br. Med. Bull. 49, 810-821). Over 100 cases of proven or suspected iatrogenic transmissions to humans have now been reported. Zobeley et al. (1999) Mol. Med. 5, 240-243 provided a model system for the sterilisation of stainless steel instruments infected with scrapie prions. It was shown that mouse-adapted scrapie prions could firmly bind to stainless steel wire, as evidenced by the finding that the wire gave rise to infection when implanted into the brain of indicator mice, even after treatment with 10% formaldehyde for 1 hour.

Usually, diagnosis in humans relies on histopathology and immunohistochemical determination. Further methods for the diagnosis of prion infection require invasive procedures such as brain or tonsil biopsies. Homogenates of these biopsies are injected into the brains of test animals such as mice. If the test animals develop clinical symptoms of prion infection then the brain of the test animal is further examined to confirm that prions are present. Problems associated with this method are that prions contained within the biopsies are subject to degradation. Consequently, infectivity is usually lost within 24 hours.

The present invention seeks to overcome the problems associated with the prior art.

### SUMMARY OF THE INVENTION

The present invention provides methods for the detection of prions in a tissue/organ or fluid therefrom. The methods use a device such as a metal wire that is contacted with the tissue/organ. Surprisingly, the device is capable of binding prions within 5 minutes. The device is then removed from contact with the tissue/organ. Suprisingly, the device is able to preserve prions against degradation for greater than 3 days. Using prior art methods, prions degrade after only 24 hours. To determine if the device is binding prions, a number of different methods can be used as discussed below. Since prions bind to the device much faster than previously known, diagnosis of prion infection is significantly quicker than prior art methods.

According to the first aspect of the present invention, there is provided a method for detecting the presence of prions in a tissue/organ; said method comprising the steps of: contacting the tissue/organ with a device, wherein said device is capable of binding prions; removing said device from contact with said tissue/organ; and determining if said device is binding prions.

According to a second aspect of the present invention, there is provided a non-invasive method for determining the presence of prions in a tissue/organ; said method comprising the steps of: contacting the tissue/organ with a device, wherein said device is capable of binding prions; removing said device from contact with said tissue/organ; and determining if said device is binding prions. Preferably, said intact tissue/organ is left at least substantially intact by said non-invasive method.

The device used in the methods of the present invention advantageously preserves prions against degradation.

Preferably, the tissue/organ is mammalian. More preferably, the tissue/organ is a livestock or a human tissue/organ.

The methods of the present invention advantageously detect prions in a tissue/organ in which prions accumulate. Preferably, the tissue/organ is selected from brain, spleen, lymph node or tonsil.

The device of the present invention may comprise one or more metals or may comprise plastic such as polystyrene, or glass. It is surprisingly disclosed herein that these materials bind prion protein. Preferably, the device of the present invention may comprise one or more metals. Preferably, the metal is any one or more of the metals selected from the group consisting of steel, stainless steel, silver, gold or combinations thereof. More preferably, the metal is stainless steel.

Advantageously, the device of the present invention may comprise one or more wires or spheres of diameter less than 5 mm, preferably less than 1 mm, preferably having dimensions as mentioned in the Examples section. Preferably, the device comprises one or more metal wires.

According to a third aspect of the present invention, we provide a method for determining if a device is binding prions comprising the steps of: contacting one or more test animals with the device; incubating the test animal(s); monitoring the test animal(s) for adverse effects or death; and optionally performing a biopsy on the test animal(s) that display adverse effects or death for evidence of prions.

Preferably, one or more devices are contacted with the test animals for 1 hour or more. More preferably, one or more devices are contacted with the test animals for 5 hours or more. More preferably, one or more devices are contacted with the test animals for more than 5 hours. Most preferably, one or more devices are contacted with the test animals permanently. No ill effects due to the device itself have been observed.

The test animal(s), which may be useful in the present invention, are preferably mammals. Preferably, the test animal(s) are mice. The test animal(s) may also include transgenic mice.

Preferably, said transgenic mice comprise one or more PrP transgene(s). More preferably, the PrP transgene(s) encode a mammalian PrP. Most preferably, the PrP transgene(s) encode a livestock or a human PrP.

According to a fourth aspect of the present invention, we provide a method for determining if a device is binding prions comprising the steps of: contacting one or more cell lines with the device; incubating the cell line(s); and assaying cell line for the presence of prions/prion protein.

The presence of prions/prion protein may be assayed by any suitable method known in the art such as by protein assay, immunoassay, Western blotting or cell blotting. Preferably, the presence of PrPSc may be detected following treatment with Proteinase K.

According to a fifth aspect, the present invention provides a method for determining if a device is binding prions by detecting said prions/prion protein directly on the surface of said device. Preferably, prions/prion protein are detected in said method using a protein assay, immunoassay or Western blotting, preferably an immunoassay.

The device used in the present invention is preferably contacted with the tissue/organ for 120 minutes or less. More preferably, the device is contacted with the tissue/organ for 30 minutes or less. Most preferably, the device is contacted with the tissue/organ for 5 minutes or less.

### ADVANTAGES

The present invention has a number of advantages. These advantages will be apparent in the following description.

By way of example, the present invention is advantageous since it provides a commercially useful method.

By way of further example, the present invention is advantageous since it provides a method for detecting the presence of prions in tissue/organ.

By way of further example, the present invention is advantageous since it provides a method of preserving prions against degradation.

By way of further example, the present invention advantageously provides for the identification of one or more agents for use in the preparation of a medicament for the treatment of prion infection.

### DETAILED DESCRIPTION OF THE INVENTION

Prion, PrPc and PrPSc

As used herein the term “prion” refers to a proteinaceous infectious particle that lacks nucleic acid.

PrPSc is a conformational isoform of PrPc (the normal form of prion protein) and is believed to be the main or only component of the prion.

In a preferred embodiment of the present invention, a tissue/organ is tested that may contain prions.

Background teachings on prions have been presented by Victor A. McKusick et al on http://www.ncbi.nlm.nih.gov/Omim. The following information concerning prions has been extracted mainly from that source.

Mutations in the prion protein gene are associated with Gerstmann-Straussler disease (GSD), Creutzfeldt-Jakob disease (CJD), and familial fatal insomnia, and aberrant isoforms of the prion protein can act as an infectious agent in these disorders as well as in kuru and in scrapie in sheep.

Prusiner (1982, 1987) suggested that prions represent a new class of infectious agent that lacks nucleic acid. (The term prion, which was devised by Prusiner (1982), comes from ‘protein infectious agent.’) The prion diseases are neurodegenerative conditions transmissible by inoculation or inherited as autosomal dominant disorders. Prusiner (1994) reviewed the pathogenesis of transmissible spongiform encephalopathies and noted that a protease-resistant isoform of the prion protein was important in the pathogenesis of these diseases. Mestel (1996) reviewed the evidence for and against—and the opinions for and against—the existence of infectious proteins.

Tagliavini et al. (1991) purified and characterized proteins extracted from amyloid plaque cores isolated from 2 patients of the Indiana kindred. They found that the major component of GSD amyloid was an 11-kD degradation product of PrP, whose N-terminus corresponded to the glycine residue at position 58 of the amino acid sequence deduced from the human PrP cDNA. In addition, amyloid fractions contained larger PrP fragments with apparently N termini and amyloid P components. Tagliavini et al. (1991) interpreted these findings as indicating that the disease process leads to proteolytic cleavage of PrP, generating an amyloidogenic peptide that polymerizes into insoluble fibrils. Since no mutations of the structural gene were found in the family, factors other than the primary structure of PrP may play a crucial role in the process of amyloid formation.

One interpretation has been that the prion is a sialoglycoprotein whose synthesis is stimulated by the infectious agent that is the primary cause of this disorder and Manuelidis et al. (1987) presented evidence suggesting that the PrP peptide is not the infectious agent in CJD. Pablos-Mendez et al. (1993) reviewed the ‘tortuous history of prion diseases’ and suggested an alternative to the idea that prions are infectious, namely, that they are cytotoxic metabolites. The authors suggested that studies of the processing of the metabolite PrP and trials of agents that enhance the appearance of this protein would be useful ways to test their hypothesis. Their model predicted that substances capable of blocking the catabolism of PrP would lead to its accumulation. Increasing PrP synthesis in transgenic mice shortens the latency in experimental scrapie. The hypothesis of Pablos-Mendez et al. (1993) suggested an intracellular derailment of the degradative rather than the synthetic pathway of PrP.

Forloni et al. (1993) found that the PrP peptide 106-126 has a high intrinsic ability to polymerize into amyloid-like fibrils in vitro. They also showed that neuronal death results from chronic exposure of primary rat hippocampal cultures to micromolar concentrations of a peptide corresponding to this peptide. They suggested that the neurotoxic effect of the peptide involves an apoptotic mechanism.

It has been suggested that the infectious, pathogenic agent of the transmissible spongiform encephalopathies is a protease-resistant, insoluble form of the PrP protein that is derived posttranslationally from the normal, protease-sensitive PrP protein (Prusiner, Beyreuther and Masters, 1994). Kocisko et al. (1994) reported the conversion of normal PrP protein to the protease-resistant PrP protein in a cell-free system composed of purified constituents. This selective conversion from the normal to the pathogenic form of PrP required the presence of preexisting pathogenic PrP. The authors showed that the conversion did not require biosynthesis of new PrP protein, its amino-linked glycosylation, or the presence of its normal glycosylphosphatidylinositol anchor. This provided direct evidence that the pathogenic PrP protein can be formed from specific protein-protein interactions between it and the normal PrP protein.

Rivera et al. (1989) described a 13-year-old male with a severe progressive neurologic disorder whose karyotype showed a pseudodicentric chromosome resulting from a telomeric fusion 15p;20p. In lymphocytes the centromeric constriction of the abnormal chromosome was always that of chromosome 20, whereas in fibroblasts both centromeres were alternately constricted. The authors suggested that centromere inactivation results from a modified conformation of the functional DNA sequences preventing normal binding to centromere-specific proteins. They also postulated that the patient's disorder, reminiscent of a spongy glioneuronal dystrophy as seen in Creutzfeldt-Jakob disease, may be secondary to the presence of a mutation in the prion protein.

Collinge et al. (1990) suggested that ‘prion disease’, whether familial or sporadic, may prove to be a more appropriate diagnostic term. An Indiana kindred with GSD disease was reported by Farlow et al. (1989) and Ghetti et al. (1989). Using PrP gene analysis in genetic prediction carries potential problems arising out of uncertainty about penetrance and the complications of presymptomatic testing in any inherited late-onset neurodegenerative disorder. Collinge et al. (1991) concluded, however, that it had a role to play in improving genetic counseling for families with inherited prion diseases, allowing presymptomatic diagnosis or exclusion of CJD or GSD in persons at risk.

Gajdusek (1991) provided a chart of the PRNP mutations found to date: 5 different mutations causing single amino acid changes and 5 insertions of 5, 6, 7, 8, or 9 octapeptide repeats. He also provided a table of 18 different amino acid substitutions that have been identified in the transthyretin gene (TTR; 176300) resulting in amyloidosis and drew a parallel between the behavior of the 2 classes of disorders.

Schellenberg et al. (1991) sought the missense mutations at codons 102, 117, and 200 of the PRNP gene, as well as the PRNP insertion mutations, which are associated with CJD and GSSD, in 76 families with Alzheimer disease, 127 presumably sporadic cases of Alzheimer disease, 16 cases of Down syndrome, and 256 normal controls; none was positive for any of these mutations. Jendroska et al. (1994) used histoblot immunostaining in an attempt to detect pathologic prion protein in 90 cases of various movement disorders including idiopathic Parkinson disease (PD; 168600), multiple system atrophy, diffuse Lewy body disease (127750), Steele-Richardson-Olszewski syndrome (260540), corticobasal degeneration, and Pick disease (172700). No pathologic prion protein was identified in any of these brain specimens, although it was readily detected in 4 controls with Creutzfeldt-Jakob disease. Perry et al. (1995) used SSCP to screen for mutations at the prion locus in 82 Alzheimer disease patients from 54 families (including 30 familial cases), as well as in 39 age-matched controls. They found a 24-bp deletion around codon 68 which removed 1 of the 5 gly-pro rich octarepeats in 2 affected sibs and 1 offspring in a late-onset Alzheimer disease family. However, the other affected individuals within the same pedigree did not share this deletion, which was also detected in 3 age-matched controls in 6 unaffected members from a late-onset Alzheimer disease family. Another octarepeat deletion was detected in 3 other individuals from the same Alzheimer disease family, of whom 2 were affected. No other mutations were found. Perry et al. (1995) concluded that there was no evidence for association between prion protein mutations and Alzheimer disease in their survey.

Hsiao et al. (1990) found no mutation in the open reading frame of the PrP gene in 3 members of the family analyzed, but Hsiao et al. (1992) later demonstrated a phel98-to-ser mutation; see 176640.0011.

Palmer and Collinge (1993) reviewed mutations and polymorphisms in the prion protein gene.

Chapman et al. (1996) demonstrated fatal insomnia and significant thalamic pathology in a patient heterozygous for the pathogenic lysine mutation at codon 200 (176640.0006) and homozygous for methionine at codon 129 of the prion protein gene. They stressed the similarity of this phenotype to that associated with mutations in codon 178 (176640.0010).

Collinge et al. (1996) investigated a wide range of cases of human prion disease to identify patterns of protease-resistant PrP that might indicate different naturally occurring prion strain types. They studied protease resistant PrP from ‘new variant’ CJD to determine whether it represents a distinct strain type that can be differentiated by molecular criteria from other forms of CJD. Collinge et al. (1996) demonstrated that sporadic CJD and iatrogenic CJD (usually due to administration of growth hormone from cadaver brain) is associated with 3 distinct patterns of protease-resistant PrP on Western blots.

Types 1 and 2 are seen in sporadic CJD and in some cases of iatrogenic CJD. A third type is seen in acquired prion diseases with a peripheral route of exposure to prions. Collinge et al. (1996) reported that ‘new variant’ CJD is associated with a unique and highly consistent appearance of protease-resistant PrP on Western blots involving a characteristic pattern of glycosylation of the PrP. Transmission of CJD to inbred mice produced a PrP pattern characteristic of the inoculated CJD. Transmission of bovine spongiform encephalopathy (BSE) prion produced a glycoform ratio pattern of PrP closely similar to that of ‘new variant’ CJD. They found that the PrP from experimental BSE in macaques and naturally acquired BSE in domestic cats showed a glycoform pattern indistinguishable from that of experimental murine BSE and ‘new variant’ CJD. The report of Collinge et al. (1996) was reviewed by Aguzzi and Weissmann (1996), who concluded that Collinge et al. (1996) had reviewed the neuropathologic and clinical features of the ‘new variant’ of CJD that was related to BSE.

Prusiner (1996) provided a comprehensive review of the molecular biology and genetics of prion diseases. Collinge (1997) likewise reviewed this topic. He recognized 3 categories of human prion diseases: (1) the acquired forms include kuru and iatrogenic CJD; (2) sporadic forms include CJD in typical and a typical forms; (3) inherited forms include familial CJD, Gerstmann-Straussler-Scheinker disease, fatal familial insomnia, and the various a typical dementias. Collinge (1997) tabulated 12 pathogenetic mutations that had been reported to that time. Noting that the ability of a protein to encode a disease phenotype represents a nonmendelian form of transmission important in biology, Collinge (1997) commented that it would be surprising if evolution had not used this method for other proteins in a range of species. He referred to the identification of prion-like mechanisms in yeast (Wickner, 1994; Ter Avanesyan et al., 1994).

Horwich and Weissman (1997) reviewed the central role of prion protein in the group of related transmissible neurodegenerative diseases. The data demonstrated that prion protein is required for the disease process, and that the conformational conversion of the prion protein from its normal soluble alpha-helical conformation to an insoluble beta-sheet state is intimately tied to the generation of disease and infectivity. They noted that much about the conversion process remains unclear.

Mallucci et al. (1999) described a large English family with autosomal dominant segregation of presenile dementia, ataxia, and other neuropsychiatric features. Diagnoses of demyelinating disease, Alzheimer disease, Creutzfeldt-Jakob disease, and Gerstmann-Straussler-Scheinker syndrome had been made in particular individuals at different times. Mallucci et al. (1999) also described an Irish family, likely to be part of the same kindred, in which diagnoses of multiple sclerosis, dementia, corticobasal degeneration, and ‘new variant’ CJD had been considered in affected individuals. Molecular studies identified the disorder as prion disease due to an ala117-to-val mutation in the PRNP gene. They emphasized the diversity of phenotypic expression seen in these kindreds and proposed that inherited prion disease should be excluded by PRNP analysis in any individual presenting with a typical presenile dementia or neuropsychiatric features and ataxia, including suspected cases of ‘new variant’ CJD. Hegde et al. (1999) demonstrated that transmissible and genetic prion diseases share a common pathway of neurodegeneration. Hegde et al. (1999) observed that the effectiveness of accumulated PrP^{Sc}, an abnormally folded isoform, in causing neurodegenerative disease depends upon the predilection of host-encoded PrP to be made in a transmembrane form, termed CtmPrP. Furthermore, the time course of PrP^{Sc} accumulation in transmissible prion disease is followed closely by increased generation of CtmPrP. Thus, the accumulation of PrP^{Sc} appears to modulate in trans the events involved in generating or metabolizing CtmPrP. Hegde et al. (1999) concluded that together these data suggested that the events of CtmPrP-mediated neurodegeneration may represent a common step in the pathogenesis of genetic and infectious prion diseases.

Pr^{P}c, the cellular, nonpathogenic isoform of PrP, is a ubiquitous glycoprotein expressed strongly in neurons. Mouillet-Richard et al. (2000) used the murine 1C11 neuronal differentiation model to search for PrP^{c}-dependent signal transduction thoursough antibody-mediated crosslinking. The 1C11 clone is a committed neuroectodermal progenitor with an epithelial morphology that lacks neuron-associated functions. Upon induction, 1C11 cells develop a neural-like morphology, and may differentiate either into serotonergic or noradrenergic cells. The choice between the 2 differentiation pathways depends on the set of inducers used. Ligation of PrP^{c} with specific antibodies induced a marked decrease in the phosphorylation level of the tyrosine kinase FYN (137025) in both serotonergic and noradrenergic cells. The coupling of PrP^{c} to FYN was dependent upon caveolin-1 (601047). Mouillet-Richard et al. (2000) suggested that clathrin (see 118960) might also contribute to this coupling. The ability of the 1C11 cell line to trigger PrPc-dependent FYN activation was restricted to its fully differentiated serotonergic or noradrenergic progenies. Moreover, the signaling activity of PrPc occurred mainly at neurites. Mouillet-Richard et al. (2000) suggested that PrP^{c} may be a signal transduction protein.

Mapping

The human gene for prion-related protein has been mapped to 20 p12-pter by a combination of somatic cell hybridization and in situ hybridization (Sparkes et al., 1986) and by spot blotting of DNA from sorted chromosomes (Liao et al., 1986). Robakis et al. (1986) also assigned the PRNP locus to 20p by in situ hybridization.

By analysis of interstitial 20p deletions, Schnittger et al. (1992) demonstrated the following order of loci: pter--PRNP--SCG1 (118920)--BMP2A (112261)--PAX1 (167411)—cen. Puckett et al. (1991) identified 5-prime of the PRNP gene a RFLP that has a high degree of heterozygosity, which might serve as a useful marker for the pter-p12 region of chromosome 20.

Riek et al. (1998) used the refined NMR structure of the mouse prion protein to investigate the structural basis of inherited human transmissible spongiform encephalopathies. In the cellular form of mouse prion protein, no spatial clustering of mutation sites was observed that would indicate the existence of disease-specific subdomains. A hydrogen bond between residues 128 and 178 provided a structural basis for the observed highly specific influence of a polymorphism at position 129 in human PRNP on the disease phenotype that segregates with the asp178-to-asn (D178N; 176640.0007) mutation. Overall, the NMR structure implied that only some of the disease-related amino acid replacements lead to reduced stability of the cellular form of PRNP, indicating that subtle structural differences in the mutant proteins may affect intermolecular signaling in a variety of different ways.

Windl et al. (1999) searched for mutations and polymorphisms in the coding region of the PRNP gene in 578 patients with suspect prion diseases referred to the German Creutzfeldt-Jakob disease surveillance unit over a period of 4.5 years. They found 40 cases with a missense mutation previously reported as pathogenic. Among these, the D178N mutation was the most common. In all of these cases, D178N was coupled with methionine at codon 129, resulting in the typical fatal familial insomnia genotype. Two novel missense mutations and several silent polymorphisms were found. In their FIG. 1, Windl et al. (1999) diagrammed the known pathogenic mutations in the coding region of PRNP.

History

Aguzzi and Brandner (1999) reviewed ‘the genetics of prions’ but raised the question of whether this is a contradiction in terms since the prion, which they defined as an enigmatic agent that causes transmissible spongiform encephalopathies, is a paradigm of nongenetic pathology. The protein-only hypothesis, originally put forward by Griffith (1967), says that prion infectivity is identical to scrapie protein (PrPSc), an abnormal form of the cellular protein, now referred to as PrPc. Replication occurs by the scrapie prion recruiting cellular prion and converting it into further scrapie prion. The newly formed scrapie prion will join the conversion cycle and lead to a chain reaction of events that results in an ever-faster accumulation of scrapie prion. This hypothesis gained widespread recognition and acceptance after Prusiner (1982) purified the pathologic protein and Weissmann and his colleagues (Oesch et al., 1985; Basler et al., 1986) cloned the gene that encodes the scrapie protein as well as its normal cellular counterpart PRNP. Even more momentum was achieved when Weissmann's group (Bueler et al., 1993) showed that genetic ablation of Prnp protects mice from experimental scrapie on exposure to prions, as predicted by the protein-only hypothesis. Aguzzi and Brandner (1999) considered the finding of linkage between familial forms of prion diseases and mutations in the prion gene to be an important landmark (Hsiao et al., 1989).

Animal Model

The structural gene for prion (Prn-p) has been mapped to mouse chromosome 2. A second murine locus, Prn-i, which is closely linked to Prn-p, determines the length of the incubation period for scrapie in mice (Carlson et al., 1986). Yet another gene controlling scrapie incubation times, symbolized Pid-1, is located on mouse chromosome 17. Scott et al. (1989) demonstrated that transgenic mice harboring the prion protein gene from the Syrian hamster, when inoculated with hamster scrapie prions, exhibited scrapie infectivity, incubation times, and prion protein amyloid plaques characteristic of the hamster. Hsiao et al. (1994) found that 2 lines of transgenic mice expressing high levels of the mutant P101L prion protein developed a neurologic illness and central nervous system pathology indistinguishable from experimental murine scrapie. Amino acid 102 in human prion protein corresponds to amino acid 101 in mouse prion protein; hence, the P101L murine mutation was the equivalent of the pro102-to-leu mutation (176640.0002) which causes Gerstmann-Straussler disease in the human. Hsiao et al. (1994) reported serial transmission of neurodegeneration to mice who expressed the P101L transgene at low levels and Syrian hamsters injected with brain extracts from the transgenic mice expressing high levels of mutant P101L prion protein. Although the high-expressing transgenic mice accumulated only low levels of infectious prions in their brains, the serial transmission of disease to inoculated recipients argued that prion formation occurred de novo in the brains of these uninoculated animals and provided additional evidence that prions lack a foreign nucleic acid.

Studies on PrP knockout mice have been reported by Bueler et al. (1994), Manson et al. (1994), and Sakaguchi et al. (1996). Sakaguchi et al. (1996) reported that the PrP knockout mice produced by them were apparently normal until the age of 70 weeks, at which point they consistently began to show signs of cerebellar ataxia. Histologic studies revealed extensive loss of Purkinje cells in the majority of cerebellar folia. Atrophy of the cerebellum and dilatation of the fourth ventricle were noted. Similar pathologic changes were not noted in the PrP knockout mice produced by Bueler et al. (1994) and by Manson et al. (1994). Sakaguchi et al. (1996) noted that the difference in outcome may be due to strain differences or to differences in the extent of the knockout within the PrP gene. Notably, in all 3 lines of PrP knockout mice described, susceptibility to prion infection was lost.

Based on their studies in PrP null mice, Collinge et al. (1994) concluded that prion protein is necessary for normal synaptic function. They postulated that inherited prion disease may result from a dominant negative effect with generation of PrP^{Sc}, the posttranslationally modified form of cellular PrP, ultimately leading to progressive loss of functional PrP (PrP^{c}). Tobler et al. (1996) reported changes in circadian rhythm and sleep in PrP null mice and stressed that these alterations show intriguing similarities with the sleep alterations in fatal familial insomnia.

Mice devoid of PrP develop normally but are resistant to scrapie; introduction of a PrP transgene restores susceptibility to the disease. To identify the regions of PrP necessary for this activity, Shmerling et al. (1998) prepared PrP knockout mice expressing PrPs with amino-proximal deletions. Surprisingly, PrP with deletion of residues 32-121 or 32-134, but not with shorter deletions, caused severe ataxia and neuronal death limited to the granular layer of the cerebellum as early as 1 to 3 months after birth. The defect was completely abolished by introducing 1 copy of a wildtype PrP gene. Shmerling et al. (1998) speculated that these truncated PrPs may be nonfunctional and compete with some other molecule with a PrP-like function for a common ligand.

Telling et al. (1996) reported observations that supported the view that the fundamental event in prion diseases is a conformational change in cellular prion protein whereby it is converted into the pathologic isoform PrP^{Sc}. They found that in fatal familial insomnia (FFI), the protease-resistant fragment of PrP^{Sc} after deglycosylation has a size of 19 kD, whereas that from other inherited and sporadic prion diseases is 21 kD. Extracts from the brains of FFI patients transmitted disease to transgenic mice expressing a chimeric human-mouse PrP gene about 200 days after inoculation and induced formation of the 19-kD PrPSc fragment, whereas extracts from the brains of familial and sporadic Creutzfeldt-Jakob disease patients produced the 21-kd) PrP^{Sc} fragment in these mice. The results of Telling et al. (1996) indicated that the conformation of PrP^{Sc} functions as a template in directing the formation of nascent PrPSc and suggested a mechanism to explain strains of prions where diversity is encrypted in the conformation of PrP^{Sc}.

Lindquist (1997) pointed out that ‘some of the most exciting concepts in science issue from the unexpected collision of seemingly unrelated phenomena.’ The case in point she discussed was the suggestion by Wickner (1994) that 2 baffling problems in yeast genetics could be explained by an hypothesis similar to the prion hypothesis. Two yeast mutations provided a convincing case that the inheritance of phenotype can sometimes be based upon the inheritance of different protein conformations rather than upon the inheritance of different nucleic acids. Thus, yeast may provide important new tools for the study of prion-like processes. Furthermore, she suggested that prions need not be pathogenic. Indeed, she suggested that self-promoted structural changes in macromolecules lie at the heart of a wide variety of normal biologic processes, not only epigenetic phenomena, such as those associated with altered chromatin structures, but also some normal, developmentally regulated events.

Hegde et al. (1998) studied the role of different topologic forms of PrP in transgenic mice expressing PrP mutations that alter the relative ratios of the topologic forms. One form is fully translocated into the ER lumen and is termed PrP-Sec. Two other forms span the ER membrane with orientation of either the carboxy-terminal to the lumen (PrP-Ctm) or the amino-terminal to the lumen (PrP-Ntm). F2-generation mice harboring mutations that resulted in high levels of PrP-Ctm showed onset of neurodegeneration at 58+/−11 days. Overexpression of PrP was not the cause. Neuropathology showed changes similar to those found in scrapie, but without the presence of PrP^{Sc}The level of expression of PrP-Ctm correlated with severity of disease.

Supattapone et al. (1999) reported that expression of a redacted PrP of 106 amino acids with 2 large deletions in transgenic (Tg) mice deficient for wildtype PrP (Pmp −/−) supported prion propagation. Rocky Mountain laboratory (RML) prions containing full-length PrP^{Sc} produced disease in Tg(PrP106)Pmp −/− mice after approximately 300 days, while transmission of RML106 prions containing PrP^{Sc106} created disease in Tg(PrP106)Prnp −/− mice after approximately 66 days on repeated passage. This artificial transmission barrier for the passage of RML prions was diminished by the coexpression of wildtype mouse PrP^{c} in Tg(PrP106)Prnp +/− mice that developed scrapie in approximately 165 days, suggesting that wildtype mouse PrP acts in trans to accelerate replication of RML106 prions. Purified PrP^{Sc106} was protease resistant, formed filaments, and was insoluble in nondenaturing detergents.

Kuwahara et al. (1999) established hippocampal cell lines from Prnp −/− and Prnp +/+ mice. The cultures were established from 14-day-old mouse embryos. All 6 cell lines studied belonged to the neuronal precursor cell lineage, although they varied in their developmental stages. Kuwahara et al. (1999) found that serum removal from the cell culture caused apoptosis in the Prnp −/− cells but not in Prnp +/+ cells. Transduction of the prion protein or the BCL2 gene suppressed apoptosis in Prnp −/− cells under serum-free conditions. Prnp −/− cells extended shorter neurites than Prnp +/+ cells, but expression of PrP increased their length. Kuwahara et al. (1999) concluded that these findings supported the idea that the loss of function of wildtype prion protein may partly underlie the pathogenesis of prion diseases. The authors were prompted to try transduction of the BCL2 gene because BCL2 had previously been shown to interact with prion protein in a yeast 2-hybrid system. Their results suggested some interaction between BCL2 and PrP in mammalian cells as well.

In scrapie-infected mice, prions are found associated with splenic but not circulating B and T lymphocytes and in the stroma, which contains follicular dendritic cells. Formation and maintenance of mature follicular dendritic cells require the presence of B cells expressing membrane-bound lymphotoxin-alpha/beta. Treatment of mice with soluble lymphotoxin-beta receptor results in the disappearance of mature follicular dendritic cells from the spleen. Montrasio et al. (2000) demonstrated that this treatment abolished splenic prion accumulation and retards neuroinvasion after intraperitoneal scrapie inoculation. Montrasio et al. (2000) concluded that their data provided evidence that follicular dendritic cells are the principal sites for prion replication in the spleen.

Chiesa et al. (1998) generated lines of transgenic mice that expressed a mutant prion protein containing 14 octapeptide repeats, the human homolog of which is associated with an inherited prion dementia. This insertion was the largest identified to that time in the PRNP gene and was associated with a prion disease characterized by progressive dementia and ataxia, and by the presence of PrP-containing amyloid plaques in the cerebellum and basal ganglia (Owen et al., 1992; Duchen et al., 1993; Krasemann et al., 1995). Mice expressing the mutant protein developed a neurologic illness with prominent ataxia at 65 or 240 days of age, depending on whether the transgene array was, respectively, homozygous or hemizygous. Starting from birth, mutant PrP was converted into a protease-resistant and detergent-insoluble form that resembled the scrapie isoform of PrP, and this form accumulated dramatically in many brain regions throughout the lifetime of the mice. As PrP accumulated, there was massive apoptosis of granule cells in the cerebellum.

Non-Invasive

As used herein, the term “non-invasive” means that the surface of a subject to be tested using the methods of the present invention is preferably not broken, punctured or cut. The term “surface” as used herein may refer to skin, whether internal or external, or may refer to surfaces such as mucosal membranes, respiratory surfaces, or the walls of anatomical surfaces such as the alimentary canal, ear canal, buccal cavity, throat or any other surface of a subject.

Preferably, the methods of the present invention are non-invasive.

Tissue/Organ

As used herein, the term “tissue/organ” refers to any tissue/organ that is to be tested for the presence of prions according to the methods of the present invention.

The tissue/organ may be or may be derived from any tissue/organ in which prions accumulate.

Preferably, the tissue/organ is a brain, spleen, lymph node or tonsil. More preferably, the tissue/organ is a brain or tonsil.

The tissue/organ may also be in the form of a biopsy or homogenate.

The tissue/organ, biopsy or homogenate may also include the fluid from said tissue/organ, which may comprise sputum, mucus or other such fluids.

As used herein, the term “intact” means that tissue or a biopsy is not removed from a subject using the devices or methods of the present invention, except possibly at de minimis levels.

Binding Prions

As used herein, the term “binding prions” refers to the adherence, association, binding, sticking, or other such interaction of prions with metal surfaces.

The binding between metals and prions may occur by any form of binding capable of occurring between metals and proteins such as covalent, ionic, Van Der Waals, transient or reversible association, or any other forms of binding interaction.

Preserving Prions

As used herein, the term “preserving prions” refers to the suprising finding disclosed in the present invention that when prions bind to metal surfaces they are preserved. As used herein, the term “preserved” means that the prions bound to the metal surface are protected against degradation and thus remain infective for a period of time that is longer than would normally be expected. For example, using prior art methods, prions injected into brain remain infective for about 24 hours only. Using the methods of the present invention, prions bound to a metal surface are advantageously preserved in barin for at least 3 days.

Advantageously, the device may be incubated at a temperature of about −20° C. to further preserve the prions. The preservation may be further enhanced by any action which helps protect prions against degradation such as preventing prions from contacting proteases or preventing prions from contacting phagocytic cells.

Device

The term “device” as used herein, refers to any device that is useful in the methods of the present invention.

The device may be any device that is capable of binding prions.

Preferably, the device comprises plastic such as polystyrene, glass or metal. Preferably, the device comprises metal. More preferably, the metal comprises one or more metals selected from the group consisting of steel, stainless steel, silver, gold or combinations thereof. Most preferably, the wire comprises stainless steel. As used herein, the term “combinations thereof” refers to alloys of two or more metals wherein at least one of the metals is selected from the group consisting of steel, stainless steel, silver or gold.

The device may also comprise two or more different metals or two or more different metal alloys.

Preferably, the device comprises one or more needles, spatula, pins, wires or spheres. More preferably, the device comprises one or more wires. Most preferably, the device comprises one or wires each measuring about 0.15 mm in diameter and 5 mm in length, such as stainless steel suture monofilament wire available from Braun MelsungerAG, Germany.

According to the methods of the present invention, the tissue/organ is contacted with the device.

Preferably, the device is sterilised before contacting the tissue/organ with the device. More preferably, the device is sterilised for 30 minutes at 11 bar (about 121° C.). Most preferably, the device is sterilised by immersing the device in 1 M NaOH for 1 hour 30 minutes at 11 bar (about 121° C.) or 4 M guanidium thiocyanate for 16 hours.

Contacting the Device

The device may be contacted with the tissue/organ such that the skin surface covering the subject is broken, punctured or cut to access said tissue/organ. Preferably, the tissue/organ is a brain, spleen, tonsil or lymph node.

Prior to contact with the device an anaesthetic such as general or a local anaesthetic may be administered to the subject if said subject is living. Alternatively, or in addition to, sedation may be administered such that the subject loses partial or total consciousness.

The methods of the present invention may comprise inserting the device into the tissue/organ such that the tissue/organ is penetrated or pierced by said device; contacting the surface of the tissue/organ with the device; contacting the device with fluid such as mucus that is associated with the tissue/organ, or any other method of contacting the tissue/organ with the device.

Non Invasive Methods

Preferably, the methods of the present invention are non-invasive. More preferably, the tissue/organ remains intact.

The tissue/organ tested using the non-invasive methods may be any tissue/organ, biopsy or homogenate in which prions accumulate.

Preferably, if a living subject is to be tested then the tissue/organ is a tonsil. This tissue/organ can be accessed via the mouth and so the skin surface covering the outside of a subject to be tested is not broken, punctured or cut.

If a living subject is to be tested, then prior to contact with the device, light sedation may be administered such that the subject does not lose consciousness. Alternatively, or in addition to, a local anaesthetic may be administered to the subject. Preferably, the anaesthetic is a local anaesthetic administered around the site of one or more tonsils.

The methods of the present invention may comprise inserting the device into the tissue/organ; contacting the surface of the tissue/organ with the device; contacting the device with fluid such as sputum or mucus or any other fluid that is associated with the issue/organ.

Preferably, the device is contacted with the tissue/organ for 120 minutes or less. More preferably, the device is contacted with the tissue/organ for 30 minutes or less. Most preferably, the device is contacted with the tissue/organ for 5 minutes or less. These times apply to both invasive and non-invasive methods of the invention.

It is an advantage of the present invention that the amount of time taken to contact the device with the tissue/organ is short. This allows results to be obtained more rapidly and more economically than other prior art methods. This also results in less discomfort or distress to the subject being tested, if said subject is living.

Removing the Device

Following contact, the device is removed from the tissue/organ or fluid therefrom. The device may be tested immediately to determine if prions are bound to it. It is an advantage of the present invention that prions are preserved when they are bound to the device. Thus, the device may be stored until it is to be tested.

Preferably, the device is stored at a temperature of about −20° C. or lower.

Testing the Device

In accordance with the present invention, the device is tested to determine if prions are bound to the surface of said device.

In one embodiment of the present invention, the device is tested by a method comprising contact with one or more test animals that are susceptible to prion infection.

In another embodiment of the present invention, the device is tested by a method comprising contact with one or more cell lines that are susceptible to prion infection.

In another embodiment, prions/prion protein are detected directly on the surface of the device. This can be done using methods such as protein assay, immunoassay or Western blotting.

Test Animal

As used herein, the term “test animal” refers to any animal that is contacted with a device to determine if the tissue/organ contains prions. The test animal can be any animal that is susceptible to infection by prions.

Preferably, the test animal is a mammal. More preferably, the test animal is an adult mammal. More preferably, the test animal is a rat, hamster, rabbit, guinea pig or mouse. Most preferably, the test animal is a mouse.

The test animal may also be a transgenic mouse such as a Tga20 mouse.

The transgenic mouse may be susceptible to prion infection by a particular strain of prion eg. a strain of prion that causes BSE in the appropriate host.

Contacting Device with test Animal

According to the present invention, the device that has been contacted with the tissue/organ is washed prior to contact with one or more test animals. Preferably, the washing step is repeated five times for 10 minutes using 50 ml of buffer per wash. Preferably, a buffer such as phosphate buffered saline is used.

The washed device is then contacted with the test animals that have been anaethetised using an anaesthetic such as halothane/O₂.

Preferably, the method of contact is via introduction of at least part of the device into the brain of the test animals, such as by inserting it directly in to the brain. More preferably, the device is inserted directly into the right parietal lobe of the brain of the test animals.

The device is contacted with the brain of one or more test animals. Preferably, the device is contacted with the brain of one or more test animals for 1 hour or less per test animal. More preferably, the device is contacted with the brain of one or more test animals for 5 hours per test animal. More preferably, the device is contacted with the brain of one or more test animals for more than 5 hours per test animal. Most preferably, the device is contacted with the brain of one or more test animals permanently.

The test animal is incubated following contact with the device. As used herein, the term “incubated” means the maintenance of the test animal in appropriate conditions, such as a containment facility as is well known in the art.

Monitoring of Test Animal

Test animals may be monitored for symptoms of prion infection by examination for the development of symptoms of prion infection. At the onset of symptoms, the test animals are examined regularly and may be culled if showing signs of distress. Criteria for clinical diagnosis of prion infection in mice are described by Carlson et al. (1986), Cell 46, 503-511 and include at least two of the following signs: generalised tremor, ataxia, rigidity of the tail, or head bobbing. Optionally, biopsies of the test animals may be performed. The biopsy may be performed on any suitable organ or tissue such as one in which prions accumulate. Preferably, a brain biopsy is performed.

Various methods well known in the art may be used for the detection of prion proteins such as Western blotting (Collinge et al. 1996, Nature 383, 685-690), immunoassay (described in WO 9837210) and electronic-property probing (described in WO 9831839).

Adverse Effects

As used herein, the term “adverse effects” refers to the clinical signs of neurological dysfunction caused by prion infection. The clinical signs of prion infection are well known in the art. When clinical signs appear, the test animals are examined daily. If the death of one or more test animals is obviously imminent, they are culled and their brains are removed for histopathologic studies and confirmation of prion infection.

Transgenic Animals

As used herein, the term “transgenic animals” refers to those animals that have one or more gene(s) in their genome that has been introduced using recombinant DNA technology. Recombinant DNA technology is well known to a person skilled in the art. In transgenic animals, the term “gene” is synonymous with the term “transgene”. The test animals of the present invention may be transgenic test animals. Preferably, said test animals may be transgenic rats, hamsters, rabbits, guinea pigs or mice. More preferably the test animals may be transgenic mice.

Exogenous PrP Genes

As used herein, the term “exogenous PrP genes” refers generally to PrP genes from any species, which encode any form of PrP amino acid sequence or protein. Some commonly known PrP sequences have been described by Gabriel et al. (1992), Proc. Natl. Acad. Sci. USA 89, 9097-9101. Accordingly, the term “exogenous PrP gene” is also used to encompass the terms “artificial PrP gene” and “chimeric PrP gene”. As used herein, the term's “artificial PrP gene” and “chimeric PrP gene” refer to genes constructed by recombinant DNA technology, using methods well known to a person skilled in the art. When exogenous PrP genes are included in the genome of an animal then it will render that animal susceptible to infection from prions that would naturally only infect a genetically distinct species. Transgenic animals containing artificial PrP genes are described in U.S. Pat. No. 5,792,901, U.S. Pat. No. 5,908,969, U.S. Pat. No. 6,008,435 and WO 9704814.

In a preferred aspect, the test animals may be mice that are transgenic for one or more exogenous PrP genes. Preferably, the exogenous PrP genes encode a mammalian PrP. Most preferably, the exogenous PrP gene(s) encode a livestock or a human PrP.

Protein Assay

According to the present invention, one or more devices may be tested for the presence of prion proteins using a protein assay. The device(s) that have been contacted with the tissue/organ are washed with a buffer. Preferably, said buffer is phosphate buffered saline. The device(s) are then incubated with proteinase K or an alkali for 1 hour at 20° C. Preferably, the alkali is 2 M NaOH. The amount of protein in the eluate is determined using a protein assay such as the Micro BCA Protein assay (Pierce, Rockford, Ill., USA) using BSA dilutions as standards.

Immunoassay

According to the present invention, the device may be tested for the presence of prions using an immunoassay. Briefly, one or more devices that have been contacted with the tissue/organ are washed with a buffer. Preferably, said buffer is phosphate buffered saline. A monoclonal antibody that is specific to the prion protein being detected is then incubated with the device. Blocking may be achieved using 5% BSA. The bound antibody can then be detected using methods such as Western blotting, Enzyme Linked Immunofiltration Assay and Enzyme Linked Immuno Sorbent Assay. Such methods are described in detail in WO 98/37210.

Cell Line

As used herein, the term “cell line” refers one or more types of cell that may be susceptible to prions. Preferably the cell line is susceptible to prions isolated from a mammal such as those prions that cause scrapie in sheep and mice. More preferably the cell line is susceptible to prions isolated from livestock or a human such as those prions that cause BSE, CJD or VCJD.

Bosque and Prusiner (2000), J. Virol. 74, 4377-4386 described a cell line called N2a that is susceptible to RML prions that cause scrapie in mice. When the N2a cell line was inoculated with RML-prion infected mouse brain homogenates, prion protein was detected using a cell blotting method after 15 days. Cultures that were negative at 20 days remained negative and so cultures were assayed 20 or more days after inoculation.

According to the present invention, one or more devices may be tested for the presence of prion proteins using one or more cell lines. Briefly, one or more devices that have been contacted with the tissue/organ are washed with a buffer. Preferably, said buffer is phosphate buffered saline. The cell line(s) are grown using methods well known in the art. Preferably, the device(s) are contacted with the cell line(s) for 1 hour or more. More preferably, the device(s) are contacted with the cell line(s) for 5 hours or more. More preferably, the device(s) are contacted with the cell line(s) for more than 5 hours. More preferably, the device(s) are contacted with the cell line(s) for 1 day or more. More preferably, the device(s) are contacted with the cell line(s) for 3 days or more. Most preferably, the device(s) are contacted with the cell line(s) for more than 3 days. The cells are cultured and after 4 days the cells are split at a 1:10 ratio in fresh medium. The presence of prion protein in the cell line is detected using various methods known in the art. Preferably, the methods used are protein assay, immunoassay, Western blotting or cell blotting. More preferably, the method used is cell blotting.

Cell Blotting

According to the present invention, the presence of prion protein in one or more cell lines that have been contacted with one or more devices may be detected by cell blotting according to Bosque and Prusiner (2000), J. Virol. 74, 4377-4386. Briefly, plastic coverslips are placed in the wells of a 24-well plate and cells are plated into the wells. After 4 days, the medium is removed and the wells are washed with a buffer such as PBS. A nitrocellulose membrane is soaked in lysis buffer and pressed firmly on to the coverslips such that the cells come into contact with the nitrocellulose membrane. The membrane is incubated with proteinase K and washed in distilled water. Next the blot is washed with denaturing buffer and blocked 5% non-fat milk and 0.1% Tween-20). The blot was then incubated with an antibody specific to the type of PrP^{Sc} being detected and the procedure performed as for Western blotting. Bosque and Prusiner (2000), J. Virol. 74, 4377-4386 reported that cell blotting is about 150-fold more sensitive than Western blotting.

Identifying an Agent

In another aspect of the present invention, a method is provided for the identification of one or more agents. At least two devices are contacted with the same tissue/organ. The devices are then removed from the tissue/organ. The amount of prions that are bound to at least one of the devices is estimated. At least one of the devices is incubated with the agent(s). Following incubation with the agent(s), the amount of prions bound to the device is estimated. The amount of prions bound to the device before and after incubation with the agent(s) is determined. Preferably, the agent(s) decrease the amount of prions bound to the device. More preferably, the agent(s) modulate prion infection.

Estimating Prion Levels

The amount of prions bound to a device may be estimated by a method such as protein assay, immunoassay, Western blotting or using cell lines and cell blotting.

Agent

As used herein, the term “agent” may be a single entity or it may be a combination of entities.

The agent may be an organic compound or other chemical. The agent may be a compound, which is obtainable from or produced by any suitable source, whether natural or artificial. The agent may be an amino acid molecule, a polypeptide, or a chemical derivative thereof, or a combination thereof. The agent may even be a polynucleotide molecule—which may be a sense or an anti-sense molecule. The agent may even be an antibody.

The agent may be designed or obtained from a library of compounds, which may comprise peptides, as well as other compounds, such as small organic molecules.

By way of example, the agent may be a natural substance, a biological macromolecule, or an extract made from biological materials such as bacteria, fungi, or animal (particularly mammalian) cells or tissues, an organic or an inorganic molecule, a synthetic agent, a semi-synthetic agent, a structural or functional mimetic, a peptide, a peptidomimetics, a derivatised agent, a peptide cleaved from a whole protein, or a peptides synthesised synthetically (such as, by way of example, either using a peptide synthesizer or by recombinant techniques or combinations thereof, a recombinant agent, an antibody, a natural or a non-natural agent, a fusion protein or equivalent thereof and mutants, derivatives or combinations thereof.

Typically, the agent will be an organic compound. Typically the organic compounds will comprise two or more hydrocarbyl groups. Here, the term “hydrocarbyl group” means a group comprising at least C and H and may optionally comprise one or more other suitable substituents. Examples of such substituents may include halo-, alkoxy-, nitro-, an alkyl group, a cyclic group etc. In addition to the possibility of the substituents being a cyclic group, a combination of substituents may form a cyclic group. If the hydrocarbyl group comprises more than one C then those carbons need not necessarily be linked to each other. For example, at least two of the carbons may be linked via a suitable element or group. Thus, the hydrocarbyl group may contain hetero atoms. Suitable hetero atoms will be apparent to those skilled in the art and include, for instance, sulphur, nitrogen and oxygen. For some applications, preferably the agent comprises at least one cyclic group. The cyclic group may be a polycyclic group, such as a non-fused polycyclic group. For some applications, the agent comprises at least the one of said cyclic groups linked to another hydrocarbyl group.

The agent may contain halo groups. Here, “halo” means fluoro, chloro, bromo or iodo.

The agent may contain one or more of alkyl, alkoxy, alkenyl, alkylene and alkenylene groups—which may be unbranched- or branched-chain.

The agent may be in the form of a pharmaceutically acceptable salt—such as an acid addition salt or a base salt—or a solvate thereof, including a hydrate thereof. For a review on suitable salts see Berge et al, J. Pharm. Sci., 1977, 66, 1-19.

The agent of the present invention may be capable of displaying other therapeutic properties.

The agent may be used in combination with one or more other pharmaceutically active agents.

If combinations of active agents are administered, then they may be administered simultaneously, separately or sequentially.

In a further aspect, the present invention also provides a method for identifying one or more agents comprising the steps of: contacting the tissue/organ with a device, wherein said device is capable of binding prions; removing said device from contact with said tissue/organ; estimating the amount of prions bound to said device; incubating agents with said device; determining if said agents decrease the amount of prions bound to the device.

Thus, in another aspect, the present invention relates to one or more agents capable of modulating prion infection. Said agent(s) may be advantageously used in the preparation of a medicament. Thus, in another aspect, the invention relates to modulation of prion infection in a subject by administering to said subject a therapeutically effective amount of said agent(s).

Amino Acid Sequence

Amino acid sequences may comprise the agent of the present invention.

As used herein, the term “amino acid sequence” is synonymous with the term “polypeptide” and/or the term “protein”. In some instances, the term “amino acid sequence” is synonymous with the term “peptide”. In some instances, the term “amino acid sequence” is synonymous with the term “protein”.

The amino acid sequence may be isolated from a suitable source, or it may be made synthetically or it may be prepared by use of recombinant DNA techniques.

Nucleotide Sequence

Nucleotide sequences may be used to express amino acid sequences that may be used as a component of the composition of the present invention.

As used herein, the term “nucleotide sequence” is synonymous with the term “polynucleotide”.

The nucleotide sequence may be DNA or RNA of genomic or synthetic or recombinant origin. The nucleotide sequence may be double-stranded or single-stranded whether representing the sense or antisense strand or combinations thereof.

The nucleotide sequence may be DNA.

The nucleotide sequence may be prepared by use of recombinant DNA techniques (e.g. recombinant DNA).

The nucleotide sequence may be cDNA.

The nucleotide sequence may be the same as the naturally occurring form, or may be derived therefrom.

Variants/Homologues/Derivatives

The present invention also encompasses the use of variants, homologues and derivatives of any thereof. Here, the term “homologue” means an entity having a certain homology with the subject amino acid sequences and the subject nucleotide sequences. Here, the term “homology” can be equated with “identity”.

In the present context, an homologous sequence is taken to include an amino acid sequence which may be at least 75, 85 or 90% identical, preferably at least 95 or 98% identical to the subject sequence. Typically, the homologues will comprise the same active sites etc. as the subject amino acid sequence. Although homology can also be considered in terms of similarity (i.e. amino acid residues having similar chemical properties/functions), in the context of the present invention it is preferred to express homology in terms of sequence identity.

In the present context, an homologous sequence is taken to include a nucleotide sequence which may be at least 75, 85 or 90% identical, preferably at least 95 or 98% identical to the subject sequence. Typically, the homologues will comprise the same sequences that code for the active sites etc. as the subject sequence. Although homology can also be considered in terms of similarity (i.e. amino acid residues having similar chemical properties/functions), in the context of the present invention it is preferred to express homology in terms of sequence identity.

Homology comparisons may be conducted by eye, or more usually, with the aid of readily available sequence comparison programs. These commercially available computer programs can calculate % homology between two or more sequences.

% homology may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example when using the GCG Wisconsin Bestfit package the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Atschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). However, for some applications, it is preferred to use the GCG Bestfit program. A new tool, called BLAST 2 Sequences is also available for comparing protein and nucleotide sequence (see FEMS Microbiol Lett 1999 174(2): 247-50; FEMS Microbiol Lett 1999 177(1): 187-8).

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). For some applications, it is preferred to use the public default values for the GCG package, or in the case of other software, the default matrix, such as BLOSUM62.

Once the software has produced an optimal alignment, it is possible to calculate % homology, preferably % sequence identity. The software typically does this as part of the sequence comparison and generates a numerical result.

The sequences may also have deletions, insertions or substitutions of amino acid residues which produce a silent change and result in a functionally equivalent substance. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, thourseonine, phenylalanine, and tyrosine.

Conservative substitutions may be made, for example according to the Table below. Amino acids in the same block in the second column and preferably in the same line in the third column may be substituted for each other:

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

The present invention also encompasses homologous substitution (substitution and replacement are both used herein to mean the interchange of an existing amino acid residue, with an alternative residue) may occur i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Replacements may also be made by unnatural amino acids include; alpha* and alpha-disubstituted* amino acids, N-alkyl amino acids*, lactic acid*, halide derivatives of natural amino acids such as trifluorotyrosine*, p-Cl-phenylalanine*, p-Br-phenylalanine*, p-1-phenylalanine*, L-allyl-glycine*, B-alanine*, L-α-amino butyric acid*, L-γ-amino butyric acid*, L-α-amino isobutyric acid*, L-ε-amino caproic acid^{#}, 7-amino heptanoic acid*, L-methionine sulfone^{#*}, L-norleucine*, L-norvaline*, p-nitro-L-phenylalanine*, L-hydroxyproline^{#}, L-thioproline*, methyl derivatives of phenylalanine (Phe) such as 4-methyl-Phe*, pentamethyl-Phe*, L-Phe (4-amino)^{#}, L-Tyr (methyl)*, L-Phe (4-isopropyl)*, L-Tic (1,2,3,4-tetrahydroisoquinoline-3-carboxyl acid)*, L-diaminopropionic acid^{#} and L-Phe (4-benzyl)*. The notation * has been utilised for the purpose of the discussion above (relating to homologous or non-homologous substitution), to indicate the hydrophobic nature of the derivative whereas # has been utilised to indicate the hydrophilic nature of the derivative, #* indicates amphipathic characteristics.

Variant amino acid sequences may include suitable spacer groups that may be inserted between any two amino acid residues of the sequence including alkyl groups such as methyl, ethyl or propyl groups in addition to amino acid spacers such as glycine or β-alanine residues. A further form of variation, involves the presence of one or more amino acid residues in peptoid form, will be well understood by those skilled in the art. For the avoidance of doubt, “the peptoid form” is used to refer to variant amino acid residues wherein the α-carbon substituent group is on the residue's nitrogen atom rather than the α-carbon. Processes for preparing peptides in the peptoid form are known in the art, for example Simon R J et al., PNAS (1992) 89(20), 9367-9371 and Horwell D C, Trends Biotechnol. (1995) 13(4), 132-134.

The nucleotide sequences for use in the present invention may include within them synthetic or modified nucleotides. A number of different types of modification to oligonucleotides are known in the art. These include methylphosphonate and phosphorothioate backbones and/or the addition of acridine or polylysine chains at the 3′ and/or 5′ ends of the molecule. For the purposes of the present invention, it is to be understood that the nucleotide sequences described herein may be modified by any method available in the art. Such modifications may be carried out in to enhance the in vivo activity or life span of nucleotide sequences useful in the present invention.

The present invention may also involve the use of nucleotide sequences that are complementary to the sequences identified using the methods presented herein, or any derivative, fragment or derivative thereof. If the sequence is complementary to a fragment thereof then that sequence can be used as a probe to identify similar coding sequences in other organisms etc.

Hybridisation

The present invention may also encompass the use of nucleotide sequences that are capable of hybridising to nucleotide sequences, or any derivative, fragment or derivative thereof—such as if the agent is an anti-sense sequence.

The term “hybridization” as used herein shall include “the process by which a strand of nucleic acid joins with a complementary strand through base pairing” as well as the process of amplification as carried out in polymerase chain reaction (PCR) technologies.

The term “variant” also encompasses sequences that are complementary to sequences that are capable of hybridising to other nucleotide sequences.

Preferably, the term “variant” encompasses sequences that are complementary to sequences that are capable of hybridising under stringent conditions (e.g. 50° C. and 0.2×SSC {1×SSC=0.15 M NaCl, 0.015 M Na₃citrate pH 7.0}) to nucleotide sequences.

More preferably, the term “variant” encompasses sequences that are complementary to sequences that are capable of hybridising under high stringent conditions (e.g. 65° C. and 0.1×SSC {1×SSC=0.15 MNaCl, 0.015 MNa₃citrate pH 7.0}) to nucleotide sequences.

Secretion

A polypeptide may be secreted from the expression host into the culture medium from where the polypeptide may be more easily recovered.

Constructs

The term “construct”—which is synonymous with terms such as “conjugate”, “cassette” and “hybrid”—may include a nucleotide sequence useful in the present invention directly or indirectly attached to a promoter. The term “fused” includes direct or indirect attachment. In some cases, the terms do not cover the natural combination of the nucleotide sequence coding for the protein ordinarily associated with the wild type gene promoter and when they are both in their natural environment.

The construct may even contain or express a marker which allows for the selection of the genetic construct in, for example, a bacterium, preferably of the genus Bacillus, such as Bacillus subtilis, or plants into which it has been transferred. Various markers exist which may be used, such as for example those encoding mannose-6-phosphate isomerase (especially for plants) or those markers that provide for antibiotic resistance—e.g. resistance to G418, hygromycin, bleomycin, kanamycin and gentamycin.

Vectors

The term “vector” includes expression vectors and transformation vectors and shuttle vectors.

The term “expression vector” means a construct capable of in vivo or in vitro expression.

The term “transformation vector” means a construct capable of being transferred from one entity to another entity—which may be of the species or may be of a different species. If the construct is capable of being transferred from one species to another—such as from an Escherichia coil plasmid to a bacterium, such as of the genus Bacillus, then the transformation vector is sometimes called a “shuttle vector”. It may even be a construct capable of being transferred from an E. coli plasmid to an Agrobacterium to a plant.

Vectors may be transformed into a suitable host cell as described below to provide for expression of a polypeptide encompassed in the present invention. Thus, in a further aspect the invention provides a process for preparing polypeptides for use in the present invention which comprises cultivating a host cell transformed or transfected with an expression vector as described above under conditions to provide for expression by the vector of a coding sequence encoding the polypeptides, and recovering the expressed polypeptides.

The vectors may be for example, plasmid, virus or phage vectors provided with an origin of replication, optionally a promoter for the expression of the said polynucleotide and optionally a regulator of the promoter.

Vectors may contain one or more selectable marker genes. The most suitable selection systems for industrial micro-organisms are those formed by the group of selection markers which do not require a mutation in the host organism. Examples of fungal selection markers are the genes for acetamidase (amdS), ATP synthetase, subunit 9 (oliC), orotidine-5′-phosphate-decarboxylase (pvrA), phleomycin and benomyl resistance (benA). Examples of non-fungal selection markers are the bacterial G418 resistance gene (this may also be used in yeast, but not in filamentous fungi), the ampicillin resistance gene (E. coli), the neomycin resistance gene (Bacillus) and the E. coli uidA gene, coding for β-glucuronidase (GUS).

Vectors may be used in vitro, for example for the production of RNA or used to transfect or transform a host cell.

Thus, polynucleotides for use in the present invention may be incorporated into a recombinant vector (typically a replicable vector), for example a cloning or expression vector. The vector may be used to replicate the nucleic acid in a compatible host cell. Thus, quantities of polynucleotides may be made by introducing a polynucleotide into a replicable vector, introducing the vector into a compatible host cell, and growing the host cell under conditions which bring about replication of the vector. The vector may be recovered from the host cell. Suitable host cells are described below in connection with expression vectors.

Genetically engineered host cells may be used to express an amino acid sequence (or variant, homologue, fragment or derivative thereof) in screening methods for the identification of agents and antagonists. Such genetically engineered host cells could be used to screen peptide libraries or organic molecules. Antagonists and agents such as antibodies, peptides or small organic molecules will provide the basis for pharmaceutical compositions. Such agents or antagonists may be administered alone or in combination with other therapeutics for the treatment of prion infection.

Expression Vectors

A nucleotide sequence may be incorporated into a recombinant replicable vector. The vector may be used to replicate and express the nucleotide sequence. Expression may be controlled using control sequences which include promoters/enhancers and other expression regulation signals. Prokaryotic promoters and promoters functional in eukaryotic cells may be used. Tissue specific or stimuli specific promoters may be used. Chimeric promoters may also be used comprising sequence elements from two or more different promoters described above. The protein produced by a host recombinant cell by expression of a nucleotide sequence may be secreted or may be contained intracellularly depending on the sequence and/or the vector used. The coding sequences can be designed with signal sequences, which direct secretion of the substance coding sequences thoursough a particular prokaryotic or eukaryotic cell membrane.

Fusion Proteins

An amino acid sequence for use in the present invention may be produced as a fusion protein, for example to aid in extraction and purification. Examples of fusion protein partners include glutathione-S-transferase (GST), 6×His, GAL4 (DNA binding and/or transcriptional activation domains) and β-galactosidase. It may also be convenient to include a proteolytic cleavage site between the fusion protein partner and the protein sequence to allow removal of fusion protein sequences. Preferably the fusion protein will not hinder the activity of the protein sequence.

The fusion protein may comprise an antigen or an antigenic determinant fused to the substance of interest. The fusion protein may be a non-naturally occurring fusion protein comprising a substance, which may act as an adjuvant in the sense of providing a generalised stimulation of the immune system. The antigen or antigenic determinant may be attached to either the amino or carboxy terminus of the substance.

An amino acid sequence may be ligated to a heterologous sequence to encode a fusion protein. For example, for screening of peptide libraries for agents capable of affecting the substance activity, it may be useful to encode a chimeric substance expressing a heterologous epitope that is recognized by a commercially available antibody.

Stereo and Geometric Isomers

The agents may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those agents, and mixtures thereof. The terms used in the claims encompass these forms, provided said forms retain the appropriate functional activity (though not necessarily to the same degree).

Pharmaceutical Salt

The agent may be administered in the form of a pharmaceutically acceptable salt.

Pharmaceutically-acceptable salts are well known to those skilled in the art, and for example include those mentioned by Berge et al, in J.Pharm.Sci., 66, 1-19 (1977). Suitable acid addition salts are formed from acids which form non-toxic salts and include the hydrochloride, hydrobromide, hydroiodide, nitrate, sulphate, bisulphate, phosphate, hydrogenphosphate, acetate, trifluoroacetate, gluconate, lactate, salicylate, citrate, tartrate, ascorbate, succinate, maleate, fumarate, gluconate, formate, benzoate, methanesulphonate, ethanesulphonate, benzenesulphonate and p-toluenesulphonate salts.

When one or more acidic moieties are present, suitable pharmaceutically acceptable base addition salts can be formed from bases which form non-toxic salts and include the aluminium, calcium, lithium, magnesium, potassium, sodium, zinc, and pharmaceutically-active amines such as diethanolamine, salts.

A pharmaceutically acceptable salt of an agent may be readily prepared by mixing together solutions of an agent and the desired acid or base, as appropriate. The salt may precipitate from solution and be collected by filtration or may be recovered by evaporation of the solvent.

An agent may exisit in polymorphic form.

An agent may contain one or more asymmetric carbon atoms and therefore exist in two or more stereoisomeric forms. Where an agent contains an alkenyl or alkenylene group, cis (E) and trans (Z) isomerism may also occur. The present invention includes the individual stereoisomers of an agent and, where appropriate, the individual tautomeric forms thereof, together with mixtures thereof.

Separation of diastereoisomers or cis- and trans-isomers may be achieved by conventional techniques, e.g. by fractional crystallisation, chromatography or H.P.L.C. of a stereoisomeric mixture of an agent or a suitable salt or derivative thereof. An individual enantiomer of an agent may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also encompasses all suitable isotopic variations of an agent or a pharmaceutically acceptable salt thereof. An isotopic variation of an agent or a pharmaceutically acceptable salt thereof is defined as one in which at least one atom is replaced by an atom having the same atomic number but an atomic mass different from the atomic mass usually found in nature. Examples of isotopes that may be incorporated into an agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of an agent and pharmaceutically acceptable salts thereof, for example, those in which a radioactive isotope such as ³H or ¹⁴C is incorporated are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of an agent of the present invention and pharmaceutically acceptable salts thereof of this invention can generally be prepared by conventional procedures using appropriate isotopic variations of suitable reagents.

It will be appreciated by those skilled in the art that an agent may be derived from a prodrug. Examples of prodrugs include entities that have certain protected group(s) and which may not possess pharmacological activity as such, but may, in certain instances, be administered (such as orally or parenterally) and thereafter metabolised in the body to form an agent of the present invention which are pharmacologically active.

It will be further appreciated that certain moieties known as “pro-moieties”, for example as described in “Design of Prodrugs” by H. Bundgaard, Elsevier, 1985 (the disclosured of which is hereby incorporated by reference), may be placed on appropriate functionalities of agents. Such prodrugs are also included within the scope of the invention.

The present invention also includes the use of zwitterionic forms of an agent of the present invention. The terms used in the claims encompass one or more of the forms just mentioned.

Solvates

The present invention also includes the use of solvate forms of an agent of the present invention.

Pro-Drug

As indicated, the present invention may also include the use of pro-drug forms of an agent.

Pharmaceutically Active Salt

An agent may be administered as a pharmaceutically acceptable salt. Typically, a pharmaceutically acceptable salt may be readily prepared by using a desired acid or base, as appropriate. The salt may precipitate from solution and be collected by filtration or may be recovered by evaporation of the solvent.

Chemical Synthesis Methods

An agent may be prepared by chemical synthesis techniques.

It will be apparent to those skilled in the art that sensitive functional groups may need to be protected and deprotected during synthesis of a compound of the invention. This may be achieved by conventional techniques, for example as described in “Protective Groups in Organic Synthesis” by T W Greene and P G M Wuts, John Wiley and Sons Inc. (1991), and by P. J. Kocienski, in “Protecting Groups”, Georg Thieme Verlag (1994).

It is possible during some of the reactions that any stereocentres present could, under certain conditions, be racemised, for example if a base is used in a reaction with a substrate having an optical centre comprising a base-sensitive group. This is possible during e.g. a guanylation step. It should be possible to circumvent potential problems such as this by choice of reaction sequence, conditions, reagents, protection/deprotection regimes, etc. as is well-known in the art.

The compounds and salts of the invention may be separated and purified by conventional methods.

Separation of diastereomers may be achieved by conventional techniques, e.g. by fractional crystallisation, chromatography or H.P.L.C. of a stereoisomeric mixture of a compound of formula (1) or a suitable salt or derivative thereof. An individual enantiomer of a compound of formula (1) may also be prepared from a corresponding optically pure intermediate or by resolution, such as by H.P.L.C. of the corresponding racemate using a suitable chiral support or by fractional crystallisation of the diastereomeric salts formed by reaction of the corresponding racemate with a suitably optically active acid or base.

An agent or variants, homologues, derivatives, fragments or mimetics thereof may be produced using chemical methods to synthesize an agent in whole or in part. For example, if they are peptides, then peptides may be synthesized by solid phase techniques, cleaved from the resin, and purified by preparative high performance liquid chromatography (e.g., Creighton (1983) Proteins Structures And Molecular Principles, WH Freeman and Co, New York N.Y.). The composition of the synthetic peptides may be confirmed by amino acid analysis or sequencing (e.g., the Edman degradation procedure; Creighton, supra).

Synthesis of peptide agents may be performed using various solid-phase techniques (Roberge J Y et al (1995) Science 269: 202-204) and automated synthesis may be achieved, for example, using the ABI 43 1 A Peptide Synthesizer (Perkin Elmer) in accordance with the instructions provided by the manufacturer. Additionally, the amino acid sequences comprising an agent or any part thereof, may be altered during direct synthesis and/or combined using chemical methods with a sequence from other subunits, or any part thereof, to produce a variant agent.

In an alternative embodiment of the invention, the coding sequence of a peptide agent (or variants, homologues, derivatives, fragments or mimetics thereof) may be synthesized, in whole or in part, using chemical methods well known in the art (see Caruthers M H et al (1980) Nuc Acids Res Symp Ser 215-23, Horn T et al (1980) Nuc Acids Res Symp Ser 225-232).

Mimetic

As used herein, the term “mimetic” relates to any chemical which includes, but is not limited to, a peptide, polypeptide, antibody or other organic chemical which has the same qualitative activity or effect as a reference agent.

Chemical Derivative

The term “derivative” or “derivatised” as used herein includes chemical modification of an agent. Illustrative of such chemical modifications would be replacement of hydrogen by a halo group, an alkyl group, an acyl group or an amino group.

Chemical Modification

The chemical modification of an agent may either enhance or reduce hydrogen bonding interaction, charge interaction, hydrophobic interaction, Van Der Waals interaction or dipole interaction between the agent and the target.

In one aspect, the identified agent may act as a model (for example, a template) for the development of other compounds.

Recombinant Methods

An agent or target may be prepared by recombinant DNA techniques.

Other Active Components

A composition may comprise other therapeutic substances in addition to the agent.

Antibody

An agent for use in the composition may comprise one or more antibodies.

The “antibody” as used herein includes but is not limited to, polyclonal, monoclonal, chimeric, single chain, Fab fragments and fragments produced by a Fab expression library. Such fragments include fragments of whole antibodies which retain their binding activity for a target substance, Fv, F(ab′) and F(ab′)₂ fragments, as well as single chain antibodies (scFv), fusion proteins and other synthetic proteins which comprise the antigen-binding site of the antibody. Furthermore, the antibodies and fragments thereof may be humanised antibodies, for example as described in U.S. Pat. No. 239,400. Neutralizing antibodies, i.e., those, which inhibit biological activity of the substance polypeptides, are especially preferred for diagnostics and therapeutics.

Antibodies may be produced by standard techniques, such as by immunisation with the substance of the invention or by using a phage display library.

If polyclonal antibodies are desired, a selected mammal (e.g., mouse, rabbit, goat, horse, etc.) is immunised with an immunogenic polypeptide bearing epitope(s) obtainable from an identified agent and/or substance of the present invention. Depending on the host species, various adjuvants may be used to increase immunological response. Such adjuvants include, but are not limited to, Freund's, mineral gels such as aluminium hydroxide, and surface-active substances such as lysolecithin, pluronic polyols, polyanions, peptides, oil emulsions, keyhole limpet hemocyanin, and dinitrophenol. BCG (Bacilli Calmette-Guerin) and Corynebacterium parvum are potentially useful human adjuvants which may be employed if purified the substance polypeptide is administered to immunologically compromised individuals for the purpose of stimulating systemic defence.

Serum from the immunised animal is collected and treated according to known procedures. If serum containing polyclonal antibodies to an epitope obtainable from an identifed agent and/or substance of the present invention contains antibodies to other antigens, the polyclonal antibodies may be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals or humans.

Monoclonal antibodies directed against particular epitopes may also be readily produced by one skilled in the art. The general methodology for making monoclonal antibodies by hybridomas is well known. Immortal antibody-producing cell lines may be created by cell fusion, and also by other techniques such as direct transformation of B lymphocytes with oncogenic DNA, or transfection with Epstein-Barr virus. Panels of monoclonal antibodies produced against orbit epitopes may be screened for various properties; i.e., for isotype and epitope affinity.

Monoclonal antibodies may be prepared using any technique which provides for the production of antibody molecules by continuous cell lines in culture. These include, but are not limited to, the hybridoma technique originally described by Koehler and Milstein (1975 Nature 256:495-497), the human B-cell hybridoma technique (Kosbor et al (1983) Immunol Today 4:72; Cote et al (1983) Proc Natl Acad Sci 80:2026-2030) and the EBV-hybridoma technique (Cole et al (1985) Monoclonal Antibodies and Cancer Therapy, Alan R Liss Inc, pp 77-96). In addition, techniques developed for the production of “chimeric antibodies”, the splicing of mouse antibody genes to human antibody genes to obtain a molecule with appropriate antigen specificity and biological activity may be used (Morrison et al (1984) Proc Natl Acad Sci 81:6851-6855; Neuberger et al (1984) Nature 312:604-608; Takeda et al (1985) Nature 314:452-454). Alternatively, techniques described for the production of single chain antibodies (U.S. Pat. No. 4,946,779) may be adapted to produce the substance specific single chain antibodies.

Antibodies may also be produced by inducing in vivo production in the lymphocyte population or by screening recombinant immunoglobulin libraries or panels of highly specific binding reagents as disclosed in Orlandi et al (1989, Proc Natl Acad Sci 86: 3833-3837), and Winter G and Milstein C (1991; Nature 349:293-299).

Antibody fragments which contain specific binding sites for the substance may also be generated. For example, such fragments include, but are not limited to, the F(ab′)₂ fragments which may be produced by pepsin digestion of the antibody molecule and the Fab fragments which may be generated by reducing the disulfide bridges of the F(ab)₂ fragments. Alternatively, Fab expression libraries may be constructed to allow rapid and easy identification of monoclonal Fab fragments with the desired specificity (Huse WD et al (1989) Science 256:1275-1281).

General Assay Techniques

Any one or more of appropriate targets—such as an amino acid sequence and/or nucleotide sequence of a prion susceptibility protein or gene—may be used for identifying an agent according to the present invention.

The target employed in such a test may be free in solution, affixed to a solid support, borne on a cell surface, or located intracellularly. The abolition of target activity or the formation of binding complexes between the target and the agent being tested may be measured.

The method of the present invention may be a screen, whereby a number of agents are tested for modulating prion infection.

Techniques for drug screening may be based on the method described in Geysen, European Patent Application 84/03564, published on Sep. 13, 1984. In summary, large numbers of different small peptide test compounds are synthesized on a solid substrate, such as plastic pins or some other surface. The peptide test compounds are reacted with a suitable target or fragment thereof and washed. Bound entities are then detected—such as by appropriately adapting methods well known in the art. A purified target may also be coated directly onto plates for use in a drug screening techniques. Alternatively, non-neutralising antibodies may be used to capture the peptide and immobilise it on a solid support.

It is expected that the methods of the present invention will be suitable for both small and large-scale screening of test compounds as well as in quantitative assays.

In one preferred aspect, the present invention relates to a method of identifying agents capable of modulating the prion infection.

Reporters

A wide variety of reporters may be used to screen for agents identified in the method of the present invention with preferred reporters providing conveniently detectable signals (eg. by spectroscopy). By way of example, a number of companies such as Pharmacia Biotech (Piscataway, N.J.), Promega (Madison, Wis.), and US Biochemical Corp (Cleveland, Ohio) supply commercial kits and protocols for assay procedures. Suitable reporter molecules or labels include those radionuclides, enzymes, fluorescent, chemiluminescent, or chromogenic agents as well as substrates, cofactors, inhibitors, magnetic particles and the like. Patents teaching the use of such labels include U.S. Pat. No. 3,817,837; U.S. Pat. No. 3,850,752; U.S. Pat. No. 3,939,350; U.S. Pat. No. 3,996,345; U.S. Pat. No. 4,277,437; U.S. Pat. No. 4,275,149 and U.S. Pat. No. 4,366,241.

Host Cells

The term “host cell” may include any cell that could comprise the target for the agent of the present invention.

Thus, a further embodiment of the present invention provides host cells transformed or transfected with a polynucleotide that is or expresses the target of the present invention. Preferably said polynucleotide is carried in a vector for the replication and expression of polynucleotides that are to be the target or are to express the target. The cells will be chosen to be compatible with the said vector and may for example be prokaryotic (for example bacterial), fungal, yeast or plant cells.

The gram-negative bacterium E. coli is widely used as a host for heterologous gene expression. However, large amounts of heterologous protein tend to accumulate inside the cell. Subsequent purification of the desired protein from the bulk of E. coli intracellular proteins can sometimes be difficult.

In contrast to E. coli, bacteria from the genus Bacillus are very suitable as heterologous hosts because of their capability to secrete proteins into the culture medium. Other bacteria suitable as hosts are those from the genera Streptomyces and Pseudomonas.

Depending on the nature of the polynucleotide encoding the polypeptide useful in the present invention, and/or the desirability for further processing of the expressed protein, eukaryotic hosts such as yeasts or other fungi may be preferred. In general, yeast cells are preferred over fungal cells because they are easier to manipulate. However, some proteins are either poorly secreted from the yeast cell, or in some cases are not processed properly (e.g. hyperglycosylation in yeast). In these instances, a different fungal host organism should be selected.

Examples of suitable expression hosts within the scope of the present invention are fingi such as Aspergillus species (such as those described in EP-A-0184438 and EP-A-0284603) and Trichoderma species; bacteria such as Bacillus species (such as those described in EP-A-0134048 and EP-A-0253455), Streptomyces species and Pseudomonas species; and yeasts such as Kluyveromyces species (such as those described in EP-A-0096430 and EP-A-0301670) and Saccharomyces species. By way of example, typical expression hosts may be selected from Aspergillus niger, Aspergillus niger var. tubigenis, Aspergillus niger var. awamori, Aspergillus aculeatis, Aspergillus nidulans, Aspergillus orvzae, Trichoderma reesei, Bacillus subtilis, Bacillus licheniformis, Bacillus amyloliquefaciens, Kluyveromyces lactis and Saccharomyces cerevisiae.

The use of suitable host cells—such as yeast, fungal and plant host cells—may provide for post-translational modifications (e.g. myristoylation, glycosylation, truncation, lapidation and tyrosine, serine or thourseonine phosphorylation) as may be needed to confer optimal biological activity on recombinant expression products of the present invention.

Organism

The term “organism” includes any organism that could comprise the target according to the present invention and/or products obtained therefrom. Examples of organisms may include a fungus, yeast or a plant.

The term “transgenic organism” in relation to the present invention jncludes any organism that comprises the target according to the present invention and/or products obtained.

Therapy

Agents identified by the method of the present invention may be used as therapeutic agents—i.e. in therapy applications.

As with the term “treatment”, the term “therapy” includes curative effects, alleviation effects, and prophylactic effects.

The therapy may be on mammals such as humans or livestock.

The therapy may be for treating conditions associated with prion infection.

Pharmaceutical Compositions

Pharmaceutical compositions useful in the present invention may comprise a therapeutically effective amount of agent(s) and pharmaceutically acceptable carrier, diluent or excipient (including combinations thereof).

Pharmaceutical compositions may be for human or animal usage in human and veterinary medicine and will typically comprise any one or more of a pharmaceutically acceptable diluent, carrier, or excipient. Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent may be selected with regard to the intended route of administration and standard pharmaceutical practice. Pharmaceutical compositions may comprise as—or in addition to—the carrier, excipient or diluent any suitable binder(s), lubricant(s), suspending agent(s), coating agent(s) or solubilising agent(s).

Preservatives, stabilizers, dyes and even flavoring agents may be provided in pharmaceutical compositions. Examples of preservatives include sodium benzoate, sorbic acid and esters of p-hydroxybenzoic acid. Antioxidants and suspending agents may be also used.

There may be different composition/formulation requirements dependent on the different delivery systems. By way of example, pharmaceutical compositions useful in the present invention may be formulated to be administered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be administered by a number of routes.

Agents may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO-A-91/11172, WO-A-94/02518 and WO-A-98/55148.

If an agent is a protein, then said protein may be prepared in situ in the subject being treated. In this respect, nucleotide sequences encoding said protein may be delivered by use of non-viral techniques (e.g. by use of liposomes) and/or viral techniques (e.g. by use of retroviral vectors) such that the said protein is expressed from said nucleotide sequence.

Administration

The term “administered” includes delivery by viral or non-viral techniques. Viral delivery mechanisms include but are not limited to adenoviral vectors, adeno-associated viral (AAV) vectos, herpes viral vectors, retroviral vectors, lentiviral vectors, and baculoviral vectors. Non-viral delivery mechanisms include lipid mediated transfection, liposomes, immunoliposomes, lipofectin, cationic facial amphiphiles (CFAs) and combinations thereof.

The components useful in the present invention may be administered alone but will generally be administered as a pharmaceutical composition—e.g. when the components are in admixture with a suitable pharmaceutical excipient, diluent or carrier selected with regard to the intended route of administration and standard pharmaceutical practice.

For example, the components may be administered (e.g. orally) in the form of tablets, capsules, ovules, elixirs, solutions or suspensions, which may contain flavouring or colouring agents, for immediate-, delayed-, modified-, sustained-, pulsed- or controlled-release applications.

If the pharmaceutical is a tablet, then the tablet may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

It is to be understood that not all of the components of the pharmaceutical need be administered by the same route. Likewise, if the composition comprises more than one active component, then those components may be administered by different routes.

If a component is administered parenterally, then examples of such administration include one or more of: intravenously, intra-arterially, intraperitoneally, intrathecally, intraventricularly, intraurethoursally, intrasternally, intracranially, intramuscularly or subcutaneously administering the component; and/or by using infusion techniques.

For parenteral administration, the component is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

As indicated, the component(s) useful in the present invention may be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A™) or 1,1,1,2,3,3,3-heptafluoropropane (HFA 227™), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Alternatively, the component(s) may be administered in the form of a suppository or pessary, or it may be applied topically in the form of a gel, hydrogel, lotion, solution, cream, ointment or dusting powder. The component(s) may also be dermally or transdermally administered, for example, by the use of a skin patch. They may also be administered by the pulmonary or rectal routes. They may also be administered by the ocular route. For ophthalmic use, the compounds may be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

For application topically to the skin, the component(s) may be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, it may be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

Dose Levels

Typically, a physician will determine the actual dosage which will be most suitable for an individual subject. The specific dose level and frequency of dosage for any particular patient may be varied and will depend upon a variety of factors including the activity of the specific compound employed, the metabolic stability and length of action of that compound, the age, body weight, general health, diet, mode and time of administration, rate of excretion, drug combination, the severity of the particular condition, and the individual undergoing therapy.

Formulation

The component(s) may be formulated into a pharmaceutical composition, such as by mixing with one or more of a suitable carrier, diluent or excipient, by using techniques that are known in the art.

Animal Test Models

In vivo models may be used to investigate and/or design therapies or therapeutic agents to modulate prion infection. The models could be used to investigate the effect of various tools/lead compounds on a variety of parameters, which are implicated in the development of or treatment of prion infection. These animal test models may be used as, or in, the method of the present invention. The animal test model will be a non-human animal test model.

General Recombinant DNA Methodology Techniques

Although in general the techniques mentioned herein are well known in the art reference may be made in particular to Sambrook et al., Molecular Cloning, A Laboratory Manual (1989) and Ausubel et al., Short Protocols in Molecular Biology (1999) 4^{th} Ed, John Wiley & Sons, Inc. PCR is described in U.S. Pat. No. 4,683,195, U.S. Pat. No. 4,800,195 and U.S. Pat. No. 4,965,188.

In another aspect of the present invention, the amount of prions in a tissue/organ may also be measured by contacting the device with a test animal. This is achieved by studying the time taken for test animals contacted with the device to show clinical symptoms and the time taken for said test animals to die. Briefly, the time at which the test animals are contacted with the device is recorded. The test animals are then monitored for the development of clinical symptoms. Criteria for clinical diagnosis of prion infection in mice are described by Carlson et al. (1986), Cell 46, 503-511. At the onset of clinical symptoms the time is recorded. The test animals are monitored again, initially on a daily basis and then, as death approaches, more frequently. When death occurs, the time is again recorded. The intervals between the onset of clinical symptoms and death are calculated. This time interval is inversely proportional to the amount of prions in the sample. The logarithms of the time intervals minus a time factor are linear functions of the logarithms of the numbers of prions in the sample. The time factor is determined by maximising the linear relationship between time interval and dose in accordance with Pruisner et al. (1982), Annals. of Neurology 11 353-358.

### EXAMPLES

The present invention is illustrated with reference to the following examples.

### Example 1

Detection of prions in a sample.

The tissue/organ is the brain of a live human that is to be tested for the presence of prions. Straight stainless steel wire is conventionally sterilised. A stereotactic frame is fixed to the subjects head and light sedation is administered. A 5 mm opening is drilled in the skull such that the brain is not exposed. Two straight stainless steel wire segments are entered at opposite sites in the opening and contacted with the brain by insertion into the brain. After 5 minutes, the wires are removed and stored overnight at −20° C. in a pre-sterilised sealed tube.

To determine if the metal wires have prions bound to them, mice are used which are susceptible to infection from prions that cause CJD in humans. The mice to be contacted with the sample to be tested are bred in an animal microbiological containment level I facility and identified by ear punching. Prior to contact with the sample, the mice are anaethetised with halothane/O₂. The wire is thawed at room temperature and contacted with the right parietal lobe of the brains of five mice by permanent insertion. The mice are then maintained in an animal microbiological containment level I facility.

The mice are monitored for adverse effects every 3 days. If clinical signs of prion infection appear, the mice are examined daily and culled if showing signs of distress. Criteria for clinical diagnosis of scrapie in mice have been described by Carlson et al. (1986), Cell 46, 503-511.

The brains of the dead mice are stored at −80° C. until prion infection is to be confirmed.

Prion infection in the dead test mice is confirmed using Western blot analysis. 10% (w/v) brain homogenates are prepared in cold lysis buffer (10 mM Tris-HCl and 10 mM EDTA, pH 7.4, 100 mM NaCl, 0.5% NP-40, 0.5% sodium deoxycholate in PBS). Insoluble material is removed by centrifugation at 3000 rpm for 5 minutes. Proteinase K digestion (50 mg/ml) is performed for 1 hour at 37° C. The reaction is terminated by the addition of Pefabloc (Boehringer) to a final concentration of 2 mM. Samples are boiled for 5 minutes in an equal volume of loading buffer (125 mM Tris-HCl, pH 6.8, 20% glycerol, 4% SDS, 0.02% bromophenol blue) before electrophoresis on 16% Tris-glycine gels. Gels are blotted onto Immobilon-P membranes, blocked in 5% Blotto (5% non-fat milk powder in PBS with 0.05% Tween-20) followed by incubation overnight with the antibodies that specifically detect CJD prions. Blots are washed in PBS, 0.05% Tween-20, and incubated with an alkaline-phosphatase conjugated anti-mouse antibody for 1 hour at room temperature. Blots are washed again and developed with a chemifluorescent substrate (Amersham) and visualised on a Storm 840 phosphoimager (Molecular Dynamics).

Thus it is demonstrated that prions are detected in a sample using the methods of the present invention.

### Example 2

Detection of prions in a sample.

The tissue/organ is a frozen brain biopsy of a dead cow that is to be tested for the presence of prions. The tissue is thawed in a microbiological containment level m facility. Five stainless steel wires each measuring 0.15 mm in diameter and 5 mm in length are sterilised by immersing in 1 M NaOH for 1 hour 30 minutes at 11 bar. When the wires are cool they are each inserted into the tissue/organ.

After 5 minutes, each wire is removed from the tonsil tissue and stored overnight at −20° C. in separate pre-sterilised sealed tubes to avoid cross contamination between the wires.

The wires are assayed for prion infectivity using test mice as in Example 1.

The brains of the dead mice are stored at −80° C. until prion infection is to be confirmed.

Western blotting is performed according to Example 1 except that monoclonal antibodies specific to prions that cause BSE in their appropriate host are used.

Thus it is demonstrated that prions are detected in a sample using the methods of the present invention.

### Example 3

### Transmission of Scrapie by Steel-Surface-Bound Prions

Introduction: Prions are unusually resistant to conventional disinfection procedures. An electrode used intracerebrally on a Creutzfeldt-Jakob disease (CJD) patient transmitted the disease to two patients in succession and finally to a chimpanzee, despite attempted disinfection. Concerns that surgical instruments may transmit variant CJD have been raised by the finding of PrPSc, a surrogate marker for infectivity, in various tissues other than brain.

Materials and Methods: Stainless steel wire was exposed to scrapie-infected brain or brain homogenate, washed exhaustively and inserted into the brain of indicator mice to measure infectivity.

Results: A contact time of 5 min with scrapie-infected mouse brain suffices to render steel wire highly infectious and insertion of infectious wire into the brain of an indicator mouse for 30 min suffices to cause disease. Infectivity bound to wires persists far longer in the brain than when injected as homogenate, which can explain the extraordinary efficiency of wire-mediated infection. No detectable amounts of PrP could be eluted with NaOH, however the presence of PrP on infectious wires was demonstrated by chemiluminescence. Several recommended sterilisation procedures inactivated wire-bound mouse prions, but exposure to 10% formaldehyde was insufficient

Conclusions: Prions are readily and tightly bound to stainless steel surfaces and can transmit scrapie to recipient mice after short exposure times. This system mimics contaminated surgical instruments and will allow an assessment of sterilisation procedures.

Overview

Prions are more resistant to inactivation than most conventional pathogens (1-4). An electrode used intracerebrally on a patient suffering from sporadic CJD (sCJD) transmitted the disease to two patients in succession and finally to a chimpanzee, despite exposure to benzene, 70% ethanol and formaldehyde vapour after each use (5, 6). Concerns that surgical instruments may transmit variant Creutzfeldt-Jakob disease (vCJD) have been raised by the finding of PrP^{Sc} not only in nervous, but also in lymphatic tissue (7-10). We examined the ability of steel surfaces to bind scrapie prions by incubating steel wires overnight with scrapie-infected brain homogenates and inserting them permanently into the brain of indicator mice. This procedure resulted in efficient transmission of disease (11).

However, long-time exposure of steel wires to brain homogenate does not reflect conditions obtaining during surgical interventions. We show that wires inserted into intact brain for as little as 5 min suffices to render the wires far more infectious than overnight exposure to brain homogenate and as infectious as 0.03 ml of 1% scrapie-infected brain homogenate injected directly into the brain. Furthermore, a contact time of 30 min was sufficient to elicit infection. Our experiments provide a model to assess the effectiveness of sterilisation procedures for steel bound prions and suggest a minimally invasive approach to assess infectivity in organs such as brain and tonsils.

Materials and Methods

Preparation of Infectious Wire

Stainless steel wire segments (diameter 0.15 mm; 5 mm length) were cut from “Stainless steel suture monofilament wire”, Art.Nr. 01614037, USP 4/0, B.Braun Melsungen AG, D-34209 Melsungen, Germany; batch 1/7502 or 1/8452). Gold wire segments (5×0.13 mm, Alfa Aesar Johnson Matthey GmbH Germany) were washed ultrasonically for 15 min in 2% Triton X-100, thoroughly rinsed in distilled water, dried at 37° C. for 1 h as described (12). Brains were homogenized in 1× Dulbecco's phosphate-buffer saline (D-PBS; Gibco BRL, Glasgow, UK) by passing through 21G and 25G needles 8 times each, to give 10% (w/v) homogenates. These were centrifuged at 1,000 rpm (Eppendorf centrifuge 5415c, Hamburg, Germany) for 5 min at room temperature and the supernatants were recovered. We have recently determined that the centrifugation step result in the loss of about 80-90% of the PrP^{Sc} present in the sample (P. Kloehn, unpublished results) so that this step is better avoided. Wires were incubated with centrifuged 10% brain homogenate in PBS for 16 h and washed 5 times 10 min in 50 ml PBS, all at room temperature. The wires were air-dried, stored at room temperature for 1 day and inserted into brain of deeply anaesthetized indicator mice, using a 25-gauge injection needle as a trocar.

Chemiluminescence of Surface-Bound PrP

Twenty stainless wire segments (0.15×5 mm) were inserted into one brain hemisphere for 5 minutes. The other hemisphere was homogenized and centrifuged as described above. Twenty stainless wire segments were incubated with 0.5 ml 10% centrifuged homogenate for 5 min at room temperature, washed five times for 10 min with 50 ml D-PBS, dried for 24 h and immediately assayed for PrP. Wires were incubated with 0.2 ml of D-PBS containing 5% non-fat dry milk (w/v; Marvel, Premier Brands UK Ltd., Wirral, Merseyside, U.K.) for 1 h with agitation. After removal of the blocking reagent, they were incubated for 1 h with 200 ng/ml of anti-PrP antibody (6H4; Prionics AG, Zürich, Switzerland) in D-PBS containing 1% non-fat dry milk and washed 3 times for 5 min with 0.2 ml of D-PBS, followed by incubation for 1 h with horseradish peroxidase-conjugated rabbit anti-mouse IgG1 (1:5000 dilution; Zymed, South San Francisco, Calif., USA). After washing 5 times for 5 min with D-PBS, the wires were exposed to 0.2 ml of SuperSignal ELISA Femto Maximum Sensitivity Substrate (Pierce, Rockford, Ill., USA) according to the manufacturer's instructions. Chemiluminescence was determined by luminometer (AutoLumat LB953; EG&G Berthold GmbH, Bad Wildbad, Germany).

Results

The ability of stainless steel surfaces to bind scrapie infectivity has been previously demonstrated by incubating steel wires (5×0.15 mm) for 16 h with 10% w/v brain homogenate of terminally scrapie-sick mice, referred to below as “standard conditions” (11). To model the exposure of surgical instruments to infected tissue more realistically, we inserted wire segments for 5, 30 or 120 min into brains of scrapie-inoculated wild-type mice culled two months before the expected appearance of scrapie symptoms. These “transiently inserted” wires were washed, dried and assayed by permanent implantation into the brain of Tga20 indicator mice (13). Incubation times of the three groups lay between 65±4 and 69±5 days (Table 1, experiment 1), showing that even the shortest exposure to scrapie-infected brain rendered wires as infectious as intracerebral inoculation with 0.03 ml of 1% homogenate of the same brain homogenate (incubation time of 68±8 days). Gold wires exposed to brain homogenate into brain also acquired infectivity (Table 1, experiment 2).

TABLE 1
Infectivity of steel or gold wires after exposure to intact brain or to brain
homogenate of scrapie-infected mice

| | | | Incubation |
|---|---|---|---|
| | | | time ± s.d. |
| Inoculation | | Sick/total | (days) |
| Experiment 1 | | | |
| Wire transiently inserted | for 5 min | 5/5 | 68 ± 2 |
| | for 30 min | 6/6 | 65 ± 4 |
| | for 120 min | 6/6 | 69 ± 5 |

| | | |
|---|---|---|
| Wire exposed to 10% brain homogenate⁺ | 7/7 | 75 ± 5 |
| Brain homogenate⁺ (1%, 0.03 ml) | 4/4 | 68 ± 8 |
| Experiment 2 | | |
| Wires exposed to homogenate | | |
| Steel wire (10%, w/v) | 4/4 | 85 ± 4 |
| Gold wire (10%, w/v) | 3/3 | 74 ± 2 |
| Steel wire (1%, w/v) | 4/4 | 86 ± 8 |
| Gold wire (1%, w/v) | 4/4 | 81 ± 6 |

⁺6.8 logLD₅₀ units/ml 10% homogenate, as determined by end point titration (23) in Tga20 mice.

For experiment 1, two C57BL/6 mice were culled 87 days after i.c. inoculation with RML, that is, about 2 months before appearance of clinical symptoms. Wires were inserted into brain for the time indicated or exposed to centrifuged 10% brain homogenate for 16 h and processed as described in the Methods section. For experiment 2, wire segments were exposed to centrifuged brain homogenate of RML-infected, terminally sick CD1 mice as described in Methods.

⁺6.8 logLD₅₀ units/ml 10% homogenate, as determined by end point titration (23) in Tga20 mice.

A second important question regards the length of time an infectious wire must contact brain tissue in order to initiate disease. Infectious wires were prepared by insertion for 5 min into the brain of an infected wild-type mouse culled one month before the expected onset of scrapie symptoms. After washing, the wires were inserted transiently into the brains of anaesthetised indicator mice. As shown in Table 2, all mice exposed to a wire for 30 min or 2 h developed symptoms after 94±10 and 100±18 days, respectively. The infectious wires, with or without subsequent exposure to brain tissue, were ultimately assayed in indicator mice and in all cases caused scrapie disease after about 70 days, showing that no detectable amounts of infectivity were lost by exposure to brain.

TABLE 2
Transient insertion of infectious wires into brains of indicat r mice

| | | Incubation time ± |
|---|---|---|
| Inoculation | Sick/total | (days) |
| Wires infected by exposure to scrapie | | |
| brain | | |
| (a) Transient insertion into indicator mice | | |
| 30 min | 4/4^{$} | 94 ± 10 |
| 120 min | 2/2^{#} | 100 ± 18^{&} |
| (b) Permanent insertion into indicator mice | | |
| Wires not previously inserted | 3/3 | 71 ± 2 |
| Wires after transient insertion for: | | |
| 30 min | 4/4 | 71 ± 3 |
| 120 min | 5/5 | 68 ± 1 |
| (c) Controls | | |
| Wires exposed to brain homogenate | 6/6 | 76 ± 3 |
| Brain homogenate (1%, 0.03 ml) | 3/3 | 69 ± 3 |

^{$}Two of 6 mice died on the day of the intervention.
^{#}Four of 6 mice died within a day of the intervention.
^{&}Incubation times were 87 and 113 days

Infectious wires were prepared by insertion for 5 min into the brain of C57B16×129Sv: [symbol] culled 121 days after e.e. inoculation with RML and washed with 50 ml PBS 5 times for 10 [symbol] Infectious wires were inserted into brains of 6 deeplyl anaesthetised Tga20 indicatior mice fo [symbol] times indicated. The recovered wires were washed with 1 ml PBS and implanted into T. [symbol] indicator mice. As controls, wires incubated with centrifuged 10% homogenate (6.8 log I. [symbol] units/ml) of the same brain and the homogenate itself were introduced into indicator mice.

Earlier experiments had shown that no detectable protein could be eluted with 2 M NaOH (<50 ng protein per wire) from wires exposed to 10% brain homogenate (11). To determine whether wires exposed to brain homogenate or to intact brain had surface-bound PrP, they were incubated with monoclonal PrP antibody 6H4 (14), followed by horseradish peroxidase-conjugated rabbit anti mouse IgG1 and chemiluminescence was measured in the presence of substrate, thereby demonstrating the chemiluminescence of surface-bound PrP on stainless steel wires exposed to brain or brain homogenates. Stainless steel wire segments were transiently inserted into brains (“dipped”) or incubated with 10% brain homogenates (homogenate) from PrP knockout mice (Prnp^{o/o}), uninfected (Tga20) and RML infected, terminally sick Tga20 mice (RML-Tga20). Wires were washed, treated with anti-PrP antibody 6H4 and horseradish peroxidase-conjugated anti-mouse IgG1 antibody, and chemiluminescence was determined.

Chemiluminescence of wires transiently inserted into infected brain of terminally sick indicator mice was about 5.5 times above reagent background. After background subtraction, the values were about 4 times higher than for wires exposed to infected brain homogenate and about 1.8 times higher than for those transiently inserted into uninfected brain. This experiment shows that PrP was bound to the wire surface; the higher chemiluminescence of the sample from infected brain is in keeping with the finding that total PrP content in terminally infected mouse brain is around 5 times higher than in uninfected controls (13, 15), due to accumulation of PrP^{Sc}. We were not able to differentiate between Prp^{C} and PrP^{Sc} on wires because proteinase K treatment abolished immunofluorescence in all cases. In an attempt to desorb PrP, we extracted 40 wire segments that had been transiently inserted into scrapie-infected brain, with 0.05 ml 2 M NaOH for 1 h, neutralised the eluate with HCl and analysed half the sample by Western blot analysis. No PrP-specific immunoreactivity was detected under conditions where 0.3 ng purified glycosylated murine PrP, dissolved in NaOH and neutralised as described above, was clearly detectable. Therefore, one wire released less than 15 pg PrP, that is less than 3×10⁸ molecules. Assuming that one logLD₅₀ unit of infectivity is associated with 10⁵ PrP^{Sc} molecules (16), one wire released less than 3000 logLD₅₀ units. Yet, the incubation time resulting from one wire is about the same as that following injection of 0.03 ml 1% brain homogenate, which corresponds to about 20'000 logLD₅₀ units. This somewhat speculative calculation suggests that the amount of PrP that could have been released from the wire surface does not readily account for the wire's infectivity, raising the question whether infectivity is due to irreversibly bound PrP^{Sc} (or PrP*)

(17) rather than to desorbed prions.

Why are wire-bound prions as infectious as concentrated homogenates? Upon intracerebral inoculation with brain homogenate, infectivity is rapidly distributed throughout the mouse (18) and after 4 days or less prions are no longer detectable in the brain (19). Perhaps wire-bound prions are more stable and can therefore act over a longer period of time. We assayed infectious wires directly or after leaving them for 1 or 5 days in brains of Prnp^{+/+} or Prnp^{o/o} mice. Table 3 shows that wires remained infectious even after residing in brain tissue for 5 days, albeit at a lower level, as evidenced by incubation times of about 90 days in indicator mice. Because wire-bound infectivity remains at a locally high concentration for 5 days or longer, it may result in a greater total exposure than injected homogenate.

TABLE 3
Infectivity of prion-coated wire after exposure to brain homogenate, PBS
or brain of uninfected mice

| | | Incubation time |
|---|---|---|
| Inoculation | Sick/total | s.d. (days) |
| Infectious wire | 3/4 | 62 ± 3 |
| Experiment 1: In vitro exposure of infectious | | |
| wire to: | | |
| (a) Pmp^{o/o} brain homogenate | | |
| Wire | 4/4 | 89 ± 3 |
| Homogenate | 1/4* | 108 |
| (b) PBS | | |
| Wire | 3/3 | 85 ± 6 |
| PBS | 0/4 | >260 |
| Experiment 2: In vivo exposure of infectious | | |
| wire to: | | |
| (a) Brain of Prnp^{+/+} mice, 1 day | | |
| Wire | 3/3 | 104 ± 20 |
| Surrounding tissue | 0/8† | >260 |
| (b) Brain of Prnp^{+/+} mice, 5 days | | |
| Wire | 2/3 | 86 ± 4 |
| Surrounding tissue | 0/8† | >260 |
| (c) Brain of Prnp^{o/o} mice, 1 day | | |
| Wire | 3/3 | 79 ± 4 |
| Surrounding tissue | 1/8†^{,}* | 101 |
| (d) Brain of Prnp^{o/o} mice, 5 days | | |
| Wire | 3/3 | 91 ± 5 |
| Surrounding tissue | 0/8† | >260 |

*Scrapie diagnosis was confirmed by histopathology or histoblotting (24)
†One of 9 mice died during or after injection.

Infections wires were prepared with centrifuged 10% brain homogenate from terminally sick CD1 [symbol] (11). For the in vitro assay (expt.1), 20 wires shaken in Eppendorf tubes for 24 h at 37 ° C., ei [symbol] with 0.2 ml freshly prepared brain homogenate (10% w/v in PBS) of uninfecte Prnp^{o/o} mice or [symbol] 0.2 ml PBS/0.1% albumin, on a thermomixer (1400 rpm). After washing with 0.2 ml of the cog [symbol] solution, wires were assayed for infectivity. Thirty-μl samples of each preparation (0.4 ml) [symbol] assayed for infectivity in Tga20 indicatior mice. For the in vivo experiment (expt.2), infectious wi [symbol] were implanted into the brain of uninfected Prnp^{o/o} (C57B16) or Prnp^{o/o} mice. After 1 and 5 da [symbol] respectively, the mice washed in 1 ml PBS and assayed. The brain samples (each about 80 mg) w [symbol] homogenised in PBS to give a 10% homogenated and centrifuged samples were injected i.e. int [symbol] indicator mice each.

The wire model provided by the present invention serves as model for the sterilisation of surgical instruments by recommended (1, 3, 20) or other procedures. In a further example, infectious wire segments were subjected to different treatments and assayed. Sodium hydroxide (1 M, 1 h) or guanidinium thiocyanate (4 M, 16 h) rendered the wires completely non-infectious to the limits of the bioassay (Table 4), however all 6 indicator mice challenged with formaldehyde-treated, prion-coated wires succumbed to scrapie after 92+8 days.

TABLE 4
Infectivity of surface-bound mouse prions after various treatments

| | | Incubation time |
|---|---|---|
| Inoculation | Sick/total | s.d. (days) |
| 1. Uninfected wires | | |
| Untreated | 0/3 | >260 |
| 2. Infectious wires | | |
| Untreated | 6/6 | 76 ± 5 |
| Sodium hydroxide (1M, 1 h, 25° C.) | 0/6 | >260 |
| Formaldehyde (10%, 1 h, 25° C.) | 6/6 | 92 ± 8 |
| Guanidinium thiocyanate (4M, 16 h, 25° C.) | 0/6 | >260 |

Infectious wires were prepared with centrifuged brain homogenates and assayed as described (11). End point titration (23) of the homogenate gave a titre of 6.75 log LD50 units/ml 10% homogenate. NaOH and formaldehyde solutions were prepared immediately prior to use; 4 M guanidinium thiocyanate was RNA Lysis buffer (#40082, Applied Biosystems, Foster City, Calif., USA). Wires were exposed to 1 ml solution and washed with 1 ml PBS four times prior to implantation.

These decontamination studies provide a model for studying decontamination of instruments used in surgery. However, it is important to note that in this Example, RML mouse prions, and a mouse-adapted scrapie isolate (21) which is less heat stable than mouse-passaged BSE (301V) or the hamster strain 263K (3, 22) were used. It is clearly desirable to conduct sterilisation experiments of surface-bound infectivity according to the present invention using CJD, vCJD and BSE prions in an appropriately sensitive host. In this Example, the area of contact between wire surface and tissue is relatively small, compared with that of surgical instruments and it is therefore desirable to use scaled-up surfaces, such as those provided by small steel beads, which could conveniently be introduced into larger indicator animals, to further support the results obtained in the mouse.

Clearly, is is advantageous to use wires “dipped” for short times into brain or tonsils instead of biopsied tissue to determine the presence of PrP^{Sc} by chemiluminescence or infectivity in an appropriate indicator mouse or susceptible cultured cell line.

### Example 4

### Intravital Assay for Prion Infectivity by Transient Insertion of Wire Segments in Brain or Spleen and Analysis in Indicator Mice

The ability of stainless steel to bind scrapie infectivity has been previously demonstrated by incubating steel wires for 16 hours with 10% brain homogenate of terminally scrapie-ill CD1 mice (Zobeley et al. 1999). We show that transient insertion of stainless steel wires into brain of RML-infected C57B16 mice (87 d.p.i.) two months before the expected appearance of scrapie symptoms for 5 minutes suffices to saturate the surface with prion infectivity. Moreover, we found prion infectivity in the spleen of C57B16 mice 49 days after intracerebrally inoculation with RML by transiently inserting wires into the spleen (Table 1). Wires were inserted into the spleen of one mouse (DNA #41682) and removed after 5 and 30 minutes. “Dipped” wires were washed with PBS under standard conditions and immediately assayed by permanent implantation into the brain of indicator mice as described in Example 3. As shown in Table 1, the incubation time was 79+7 and 82+3 days, respectively. In addition, wires were transiently inserted into the whole brain of the same mouse and analysed under the same conditions. Wires exposed to the brain for 5 and 30 min caused disease in indicator mice after 87+5 (4/5) and 103+15 (3/5) days, respectively. A 1% homogenate of the same brain, transmitted disease to all indicator mice in 85+6 (5/5) days (Table 1); the titre by endpoint titration was about 4.5 logLD₅₀ units/ml.

TABLE 1
Infectivity f stainless steel wire segments exp sed to intact brain or spleen f
C57B16 mice 49 days after RML in culation.

| | | incubation time (days + |
|---|---|---|
| Inoculum | sick/total | S.D.) |
| Wire exposed to brain for 5 | 4/5+ | 87 ± 5 |
| min | | |
| Wire exposed to brain for 30 | 3/5# | 103 ± 15 |
| min | | |
| Wire exposed to spleen for 5 | 4/4 | 79 ± 7 |
| min | | |
| Wire exposed to spleen for 30 | 4/5 | 82 ± 3 |
| min | | |
| Brain homogenate (1%) | 5/5 | 85 ± 6 |
| Brain homogenate (0.01%) | 0/5 | >150 |
| Brain homogenate (0.001%) | 0/5 | >150 |
| Brain homogenate (0.0001%) | 0/5 | >150 |

+The fifth mouse developed behavioural abnormalities after 135 days (DNA#42988).
#The fourth mouse developed behavioural abnormalities after 143 days (DNA#43O91).

The dipping experiment was performed as described in Example 3.

### REFERENCES TO EXAMPLE 3

1. Ernst, D. R. & Race, R. E. (1993) J. Virol. Methods 41, 193-201.

2. Taylor, D. M., Fraser, H., McConnell, I., Brown, D. A., Brown, K. L., Lamza, K. A. & Smith, G. R. (1994) Arch. Virol. 139, 313-326.

3. Taylor, D. M. (2000) Vet. J. 159,10-17.

4. Brown, P., Gibbs, C. J., Jr., Amyx, H. L., Kingsbury, D. T., Rohwer, R. G., Sulima, M. P. & Gajdusek, D. C. (1982) N. Engl. J Med 306, 1279-1282.

5. Bernoulli, C., Siegfried, J., Baumgartner, G., Regli, F., Rabinowicz, T., Gajdusek, D. C. & Gibbs, C. J., Jr. (1977) Lancet 1, 478-479.

6. Gibbs, C. J., Jr., Asher, D. M., Kobrine, A., Amyx, H. L., Sulima, M. P. & Gajdusek, D. C. (1994) J. Neurol. Neurosurg. Psychiatry 57, 757-758.

7. Hill, A. F., Zeidler, M., Ironside, J. & Collinge, J. (1997) Lancet 349,99-100.

8. Hill, A. F., Desbruslais, M., Joiner, S., Sidle, K. C. L., Gowland, I., Collinge, J., Doey, L. J. & Lantos, P. (1997) Nature 389, 448-450.

9. Hill, A. F., Butterworth, R. J., Joiner, S., Jackson, G., Rossor, M. N., Thomas, D. J., Frosh, A., Tolley, N., Bell, J. E., Spencer, M., King, A., Al-Sarraj, S., Ironside, J. W., Lantos, P. L. & Collinge, J. (1999) Lancet 353, 183-189.

10. Wadsworth, J. D. F., Joiner, S., Hill, A. F., Campbell, T. A., Desbruslais, M. & Collinge, S. (2001) Lancet In press.

11. Zobeley, E., Plechsig, E., Cozzio, A., Enari, M. & Weissmann, C. (1999) Mol. Med. 5, 240-243.

12. Williams, R. L. & Williams, D. F. (1988) Biomaterials 9,206-212.

13. Fischer, M., Rülicke, T., Raeber, A., Sailer, A., Moser, M., Oesch, B., Brandner, S., Aguzzi, A. & Weissmann, C. (1996) EMBO J. 15,1255-1264.

14. Korth, C., Stierli, B., Streit, P., Moser, M., Schaller, O., Fischer, R., Schulz-Schaeffer, W., Kretzschmar, H., Raeber, A., Braun, U., Ehrensperger, F., Hornemann, S., Glockshuber, R., Riek, R., Billeter, M., Wuthrich, K. & Oesch, B. (1997) Nature 390, 74-77.

15. Büeler, H., Raeber, A., Sailer, A., Fischer, M., Aguzzi, A. & Weissmann, C. (1994) Mol. Med. 1, 19-30.

16. Bolton, D. C., Rudelli, R. D., Currie, J. R. & Bendheim, P. E. (1991) J. Gen. Virol. 72, 2905-2913.

17. Weissmann, C. (1991) Nature 349, 569-571.

18. Millson, G. C., Kimberlin, R. H., Manning, E. J. & Collis, S. C. (1979) Vet. Microbiol. 1979, 89-99.

19. Büeler, H., Aguzzi, A., Sailer, A., Greiner, R. A., Autenried, P., Aguet, M. & Weissmann, C. (1993) Cell 73,1339-1347.

20. Manuelidis, L. (1997) J. Neurovirol. 3, 62-65.

21. Chandler, R. L. (1961) Lancet 1, 1378-1379.

22. Taylor, D. M., Fernie, K., McConnell, I. & Steele, P. J. (1998) Vet. Microbiol. 64, 33-38.

23. Reed, J. & Muench, H. (1938) Am. J. Hygiene 27,493-497.

24. Flechsig, E., Shmerling, D., Hegyi, I., Raeber, A. J., Fischer, M., Cozzio, A., von Mering, C., Aguzzi, A. & Weissmann, C. (2000) Neuron 27, 399-408.

### PRION REFERENCES

Aguzzi, A.: Personal Communication. Zurich, Switzerland, Mar. 20, 1997.

Aguzzi, A.; Brandner, S. The genetics of prions—a contradiction in terms? Lancet 354: 22-25, 1999.

Aguzzi, A.; Weissmann, C. A suspicious signature. Nature 383: 666-667, 1996.

Basler, K.; Oesch, B.; Scott, M.; Westaway, D.; Walchli, M.; Groth, D. F.; McKinley, M. P.; Prusiner, S. B.; Weissmann, C. Scrapie and cellular PrP isoforms are encoded by the same chromosomal gene. Cell 46: 417-428, 1986.

Bertoni, J. M.; Brown, P.; Goldfarb, L. G.; Rubenstein, R.; Gajdusek, D. C. Familial Creutzfeldt-Jakob disease (codon 200 mutation) with supranuclear palsy. J.A.M.A. 268: 2413-2415, 1992.

Beyreuther, K.; Masters, C. L. Catching the culprit prion. Nature 370: 419-420, 1994.

Bosque, P. J.; Vnencak-Jones, C. L.; Johnson, M. D.; Whitlock, J. A.; McLean, M. J. A PrP gene codon 178 base substitution and a 24-bp interstitial deletion in familial Creutzfeldt-Jakob disease. Neurology 42: 1864-1870, 1992.

Brown, P.; Galvez, S.; Goldfarb, L. G.; Nieto, A.; Cartier, L.; Gibbs, C. J., Jr.; Gajdusek, D. C. Familial Creutzfeldt-Jakob disease in Chile is associated with the codon 200 mutation of the PRNP amyloid precursor gene on chromosome 20. J. Neurol. Sci. 112: 65-67, 1992.

Brown, P.; Goldfarb, L. G.; Gajdusek, D. C. The new biology of spongiform encephalopathy: infectious amyloidoses with a genetic twist. Lancet 337: 1019-1022, 1991.

Brown, P.; Goldfarb, L. G.; Kovanen, J.; Haltia, M.; Cathala, P.; Sulima, M.; Gibbs, C. J., Jr.; Gajdusek, D. C. Phenotypic characteristics of familial Creutzfeldt-Jakob disease associated with the codon 178-asn PRNP mutation. Ann. Neurol. 31: 282-285, 1992.

Brown, P.; Goldfarb, L. G.; McCombie, W. R.; Nieto, A.; Squillacote, D.; Sheremata, W.; Little, B. W.; Godec, M. S.; Gibbs, C. J., Jr.; Gajdusek, D. C.: A typical Creutzfeldt-Jakob disease in an American family with an insert mutation in the PRNP amyloid precursor gene. Neurology 42: 422-427, 1992.

Bueler, H.; Aguzzi, A.; Sailer, A.; Greiner, R-A.; Autenried, P.; Aguet, M.; Weissman, C. Mice devoid of PrP are resistant to scrapie. Cell 73: 1339-1347, 1993.

Bueler, H.; Raeber, A.; Sailer, A.; Fischer, M.; Aguzzi, A.; Weissmann, C.: High prion and PrP(Sc) levels but delayed onset of disease in scrapie-inoculated mice heterozygous for a disrupted PrP gene. Molec. Med. 1: 19-30, 1994.

Campbell, T. A.; Palmer, M. S.; Will, R. G.; Gibb, W. R. G.; Luthert, P. J.; Collinge, J. A prion disease with a novel 96-base pair insertional mutation in the prion protein gene. Neurology 46: 761-766, 1996.

Carlson, G. A.; Kingsbury, D. T.; Goodman, P. A.; Coleman, S.; Marshall, S. T.; DeArmond, S.; Westaway, D.; Prusiner, S. B. Linkage of prion protein and scrapie incubation time genes. Cell 46: 503-511, 1986.

Chapman, J.; Arlazoroff, A.; Goldfarb, L. G.; Cervenakova, L.; Neufeld, M. Y.; Werber, E.; Herbert, M.; Brown, P.; Gajdusek, D. C.; Korcyn, A. D. Fatal insomnia in a case of familial Creutzfeldt-Jakob disease with the codon 200(lys) mutation. Neurology 46: 758-761, 1996.

Chapman, J.; Ben-Israel, J.; Goldhammer, Y.; Korczyn, A. D. The risk of developing Creutzfeldt-Jakob disease in subjects with the PRNP gene codon 200 point mutation. Neurology 44: 1683-1686, 1994.

Chapman, J.; Brown, P.; Rabey, J. M.; Goldfarb, L. G.; Inzelberg, R.; Gibbs, C. J., Jr.; Gajdusek, D. C.; Korczyn, A. D. Transmission of spongiform encephalopathy from a familial Creutzfeldt-Jakob disease patient of Jewish Libyan origin carrying the PRNP codon 200 mutation. Neurology 42: 1249-1250, 1992.

Chapman, J.; Korcyn, A. D. Genetic and environmental factors determining the development of Creutzfeldt-Jakob disease in Libyan Jews. Neuroepidemiology 10: 228-231, 1991.

Chiesa, R.; Drisaldi, B.; Quaglio, E.; Migheli, A.; Piccardo, P.; Ghetti, B.; Harris, D. A. Accumulation of protease-resistant prion protein (PrP) and apoptosis of cerebellar granule cells in transgenic mice expressing a PrP insertional mutation. Proc. Nat. Acad. Sci. 97: 5574-5579, 2000.

Chiesa, R.; Piccardo, P.; Ghetti, B.; Harris, D.: Neuron 21: 1339-1351, 1998.

Collinge, J.: Human prion diseases and bovine spongiform encephalopathy (BSE). Hum. Mol. Genet. 6: 1699-1705, 1997.

Collinge, J.; Brown, J.; Hardy, J.; Mullan, M.; Rossor, M. N.; Baker, H.; Crow, T. J.; Lofthouse, R.; Poulter, M.; Ridley, R.; Owen, F.; Bennett, C.; Dunn, G.; Harding, A. E.; Quinn, N.; Doshi, B.; Roberts, G. W.; Honavar, M.; Janota, I.; Lantos, P. L.: Inherited prion disease with 144 base pair gene insertion. 2. Clinical and pathological features. Brain 115: 687-710, 1992.

Collinge, J.; Harding, A. E.; Owen, F.; Poulter, M.; Lofthouse, R.; Boughey, A. M.; Shah, T.; Crow, T. J.: Diagnosis of Gerstmann-Straussler syndrome in familial dementia with prion protein gene analysis. Lancet 11: 15-17, 1989.

Collinge, J.; Owen, F.; Poulter, M.; Leach, M.; Crow, T. J.; Rossor, M. N.; Hardy, J.; Mullan, M. J.; Janota, I.; Lantos, P. L.: Prion dementia without characteristic pathology. Lancet336: 7-9, 1990.

Collinge, J.; Palmer, M. S.; Dryden, A. J.: Genetic predisposition to iatrogenic Creutzfeldt-Jakob disease. Lancet 337: 1441-1442, 1991.

Collinge, J.; Poulter, M.; Davis, M. B.; Baraitser, M.; Owen, F.; Crow, T. J.; Harding, A. E.: Presymptomatic detection or exclusion of prion protein gene defects in families with inherited prion diseases. Am. J. Hum. Genet. 49: 1351-1354, 1991.

Collinge, J.; Sidle, K. C. L.; Heads, J.; Ironside, J.; Hill, A. F.: Molecular analysis of prion strain variation and the aetiology of ‘new variant’ CJD. Nature 383: 685-690, 1996.

Collinge, J.; Whittington, M.; Sidle, K. C. L.; Smith, C. J.; Palmer, M.; Clarke, A. R.; Jefferys, J. G. R.: Prion protein is necessary for normal synaptic function. (Letter) Nature 370: 295-297, 1994.

Colombo, R.: Age and origin of the PRNP E200K mutation causing familial Creutzfeldt-Jacob disease in Libyan Jews. (Letter) Am. J. Hum. Genet. 67: 528-531,2000.

de Silva, R.; Ironside, J. W.; McCardle, L.; Esmonde, T.; Bell, J.; Will, R.; Windl, O.; Dempster, M.; Estibeiro, P.; Lathe, R.: Neuropathological phenotype and ‘prion protein’ genotype correlation in sporadic Creutzfeldt-Jakob disease. Neurosci. Lett. 179: 50-52, 1994.

Deslys, J.-P.; Jaegly, A.; d'Aignaux, J. H.; Mouthon, F.; de Villemeur, T. B.; Dormont, D. Genotype at codon 129 and susceptibility to Creutzfeldt-Jakob disease. Lancet 351: 1251 only, 1998.

Dlouhy, S. R.; Hsiao, K.; Farlow, M. F.; Foroud, T.; Conneally, P. M.; Johnson, P.; Prusiner, S. B.; Hodes, M. E.; Ghetti, B.: Linkage of the Indiana kindred of Gerstmann-Straussler-Scheinker disease to the prion protein gene. Nature Genet. 1: 64-67, 1992.

Doh-ura, K.; Kitamoto, T.; Sakaki, Y.; Tateishi, J.: CJD discrepancy. (Letter) Nature 353: 801-802, 1991.

Doh-ura, K.; Tateishi, J.; Sasaki, H.; Kitamoto, T.; Sakaki, Y.: Pro-to-leu change at position 102 of prion protein is the most common but not the sole mutation related to Gerstmann-Straussler syndrome. Biochem. Biophys. Res. Commun. 163: 974-979, 1989.

Duchen, L. W.; Poulter, M.; Harding, A. E.: Brain 116: 555-567, 1993.

Farlow, M. R.; Yee, R. D.; Dlouhy, S. R.; Conneally, P. M.; Azzarelli, B.; Ghetti, B. Gerstmann-Straussler-Scheinker disease. I. Extending the clinical spectrum. Neurology 39: 1446-1452, 1989.

Forloni, G.; Angeretti, N.; Chiesa, R.; Monzani, E.; Salmona, M.; Bugiani, 0.: Neurotoxicity of a prion protein fragment. (Letter) Nature 362: 543-546, 1993.

Gabizon, R.; Rosemann, H.; Meiner, Z.; Kahana, I.; Kahana, E.; Shugart, Y.; Ott, J.; Prusiner, S. B.: Mutation and polymorphism of the prion protein gene in Libyan Jews with Creutzfeldt-Jakob disease (CJD). Am. J. Hum. Genet. 53: 828-835, 1993.

Gajdusek, D. C.: The transmissible amyloidoses: genetical control of spontaneous generation of infectious amyloid proteins by nucleation of configurational change in host precursors: kuru-CJD-GSS-scrapie-BSE. Europ. J. Epidemiol. 7: 567-577, 1991.

Gambetti, P.; Petersen, R.; Monari, L.; Tabaton, M.; Autilio-Gambetti, L.: Fatal familial insomnia and the widening spectrum of prion diseases. Brit. Med. Bull. 49: 980-994, 1993.

Ghetti, B.; Tagliavini, F.; Masters, C. L.; Beyreuther, K.; Giaccone, G.; Verga, L.; Farlow, M. R.; Conneally, P. M.; Dlouhy, S. R.; Azzarelli, B.; Bugiani, O.: Gerstmann-Straussler-Scheinker disease. II. Neurofibrillary tangles and plaques with PrP-amyloid coexist in an affected family. Neurology 39: 1453-1461, 1989.

Giaccone, G.; Verga, L.; Bugiani, O.; Frangione, B.; Serban, D.; Prusiner, S. B.; Farlow, M. R; Ghetti, B.; Tagliavini, F.: Prion protein preamyloid and amyloid deposits in Gerstmann-Straussler-Scheinker disease, Indiana kindred. Proc. Nat Acad. Sci. 89: 9349-9353, 1992.

Goldfarb, L. G.; Brown, P.; Cervenakova, L.; Gajdusek, D. C.: Molecular genetic studies of Creutzfeldt-Jakob disease. Molec. Neurobiol. 8: 89-97,1994.

Goldfarb, L. G.; Brown, P.; Goldgaber, D.; Asher, D. M.; Rubenstein, R.; Brown, W. T.; Piccardo, P.; Kascsak, R. J.; Boellaard, J. W.; Gajdusek, D. C.: Creutzfeldt-Jakob disease and kuru patients lack a mutation consistently found in the Gerstmann-Straussler-Scheinker syndrome. Exp. Neurol. 108: 247-250, 1990.

Goldfarb, L. G.; Brown, P.; Haltia, M.; Cathala, F.; McCombie, W. R.; Kovanen, J.; Cervenakova, L.; Goldin, L.; Nieto, A.; Godec, M. S.; Asher, D. M.; Gajdusek, D. C. Creutzfeldt-Jakob disease cosegregates with the codon 178-asn PRNP mutation in families of European origin. Ann. Neurol. 31: 274-281, 1992.

Goldfarb, L. G.; Brown, P.; McCombie, W. R.; Goldgaber, D.; Swergold, G. D.; Wills, P. R.; Cervenakova, L.; Baron, H.; Gibbs, C. J., Jr.; Gajdusek, D. C.: Transmissible familial Creutzfeldt-Jakob disease associated with five, seven, and eight extra octapeptide coding repeats in the PRNP gene. Proc. Nat. Acad. Sci. 88: 10926-10930, 1991.

Goldfarb, L. G.; Brown, P.; Mitrova, E.; Cervenakova, L.; Goldin, L.; Korczyn, A. D.; Chapman, J.; Galvez, S.; Cartier, L.; Rubenstein, R.; Gajdusek, D. C.: Creutzfeldt-Jacob disease associated with the PRNP codon 200-lys mutation: an analysis of 45 families. Europ. J. Epidemiol. 7: 477-486, 1991.

Goldfarb, L. G.; Haltia, M.; Brown, P.; Nieto, A.; Kovanen, J.; McCombie, W. R.; Trapp, S.; Gajdusek, D. C.: New mutation in scrapie amyloid precursor gene (at codon 178) in Finnish Creutzfeldt-Jakob kindred. (Letter) Lancet 337: 425, 1991.

Goldfarb, L. G.; Mitrova, E.; Brown, P.; Toh, B. H.; Gajdusek, D. C.: Mutation in codon 200 of scrapie amyloid protein gene in two clusters of Creutzfeldt-Jakob disease in Slovakia. (Letter) Lancet 336: 514-515, 1990.

Goldfarb, L. G.; Petersen, R. B.; Tabaton, M.; Brown, P.; LeBlanc, A. C.; Montagna, P.; Cortelli, P.; Julien, J.; Vital, C.; Pendelbury, W. W.; Haltia, M.; Wills, P. R.; Hauw, J. J.; McKeever, P. E.; Monari, L.; Schoursank, B.; Swergold, G. D.; Autilio-Gambetti, L.; Gajdusek, D. C.; Lugaresi, E.; Gambetti P.: Fatal familial insomnia and familial Creutzfeldt-Jakob disease: disease phenotype determined by a DNA polymorphism. Science 258: 806-808, 1992.

Goldgaber, D.; Goldfarb, L. G.; Brown, P.; Asher, D. M.; Brown, W. T.; Lin, S.; Teener, J. W.; Feinstone, S. M.; Rubenstein, R.; Kascsak, R. J.; Boellaard, J. W.; Gajdusek, D. C. Mutations in familial Creutzfeldt-Jakob disease and Gerstmann-Straussler-Scheinker's syndrome. Exp. Neurol. 106: 204-206, 1989.

Goldhammer, Y.; Gabizon, R.; Meiner, Z.; Sadeh, M.: An Israeli family with Gerstmann-Straussler-Scheinker disease manifesting the codon 102 mutation in the prion protein gene. Neurology 43: 2718-2719, 1993.

Griffith, J. S.: Self-replication and scrapie. Nature 215: 1043-1044, 1967. Haltia, M.; Kovanen, J.; Goldfarb, L. G.; Brown, P.; Gajdusek, D. C.: Familial Creutzfeldt-Jakob isease in Finland: epidemiological, clinical, pathological and molecular genetic studies. Europ. J. Epidemiol. 7: 494-500, 1991.

Hegde, R. S.; Mastrianni, J. A.; Scott, M. R.; DeFea, K. A.; Tremblay, P.; Torchia, M.; DeArmond, S. J.; Prusiner, S. B.; Lingappa, V. R.: A transmembrane form of the prion protein in neurodegenerative disease. Science 279:827-834, 1998.

Hegde, R. S.; Tremblay, P.; Groth, D.; DeArmond, S. J.; Prusiner, S. B.; Lingappa, V. R. Transmissible and genetic prion diseases share a common pathway of neurodegeneration. Nature 402: 732-736, 1999.

Horwich, A. L.; Weissman, J. S.: Deadly conformations—protein misfolding in prion disease. Cell 89: 499-510, 1997.

Hsiao, K.; Baker, H. F.; Crow, T. J.; Poulter, M.; Owen, F.; Terwilliger, J. D.; Westaway, D.; Ott, J.; Prusiner, S. B.: Linkage of a prion protein missense variant to Gerstmann-Straussler syndrome. Nature 338: 342-345, 1989.

Hsiao, K.; Cass, C.; Conneally, P. M.; Dlouhy, S. R.; Hodes, M. E.; Farlow, M. R.; Ghetti, B.; Prusiner, S. B.: A typical Gerstmann-Straussler-Scheinker syndrome with neurofibrillary tangles: no mutation in the prion protein open-reading-frame in a patient of the Indiana kindred. (Abstract) Neurobiol. Aging 11: 302, 1990.

Hsiao, K.; Dlouhy, S. R.; Farlow, M. R.; Cass, C.; Da Costa, M.; Conneally, P. M.; Hodes, M. E.; Ghetti, B.; Prusiner, S. B.: Mutant prion proteins in Gerstmann-Straussler-Scheinker disease with neurofibrillary tangles. Nature Genet. 1: 68-71, 1992.

Hsiao, K.; Meiner, Z.; Kahana, E.; Cass, C.; Kahana, I.; Avrahami, D.; Scarlato, G.; Abramsky, O.; Prusiner, S. B.; Gabizon, R.: Mutation of the prion protein in Libyan Jews with Creutzfeldt-Jakob disease. New Eng. J. Med. 324: 1091-1097, 1991.

Hsiao, K. K.; Groth, D.; Scott, M.; Yang, S.-L.; Serban, H.; Rapp, D.; Foster, D.; Torchia, M.; DeArmond, S. J.; Prusiner, S. B.: Serial transmission in rodents of neurodegeneration from transgenic mice expressing mutant prion protein. Proc. Nat. Acad. Sci. 91: 9126-9130, 1994.

Ironside, J. W.; Sutherland, K.; Bell, J. E.; McCardle, L.; Barrie, C.; Estebeiro, K.; Zeidler, M.; Will, R. G.: A new variant of Creutzfeldt-Jakob disease: neuropathological and clinical features. Cold Spring Harbor Symp. Quant Biol. 61: 523-530, 1996.

Jendroska, K.; Hoffmann, O.; Schelosky, L.; Lees, A. J.; Poewe, W.; Daniel, S. E.: Absence of disease related prion protein in neurodegenerative disorders presenting with Parkinson's syndrome. J. Neurol. Neurosurg. Psychiat. 57: 1249-1251, 1994.

Kitamoto, T.; Ohta, M.; Doh-ura, K.; Hitoshi, S.; Terao, Y.; Tateishi, J.: Novel missense variants of prion protein in Creutzfeldt-Jakob disease or Gerstmann-Straussler syndrome. Biochem. Biophys. Res. Commun. 191: 709-714, 1993.

Kocisko, D. A.; Come, J. H.; Priola, S. A.; Chesebro, B.; Raymond, G. J.; Lansbury, P. T.; Caughey, B.: Cell-free formation of protease-resistant prion protein. Nature 370: 471-474, 1994.

Krasemann, S.; Zerr, I.; Weber, T.; Poser, S.; Kretzschmar, H.; Hunsmann, G.; Bodemer, W. :Prion disease associated with a novel nine octapeptide repeat insertion in the PRNP gene. Molec. Brain Res. 34: 173-176, 1995.

Kretzschmar, H. A.; Neumann, M.; Stavrou, D.: Codon 178 mutation of the human prion protein gene in a German family (Backer family): sequencing data from 72-year-old celloidin-embedded brain tissue. Acta Neuropath. 89: 96-98, 1995.

Kretzschmar, H. A.; Stowring, L. E.; Westaway, D.; Stubblebine, W. H.; Prusiner, S. B.; DeArmond, S. J.: Molecular cloning of a human prion protein cDNA. DNA 5: 315-324, 1986.

Kuwahara, C.; Takeuchi, A. M.; Nishimura, T.; Haraguchi, K.; Kubosaki, A.; Matsumoto, Y.; Saeki, K.; Matsumoto, Y.; Yokoyama, T.; Itohara, S.; Onodera, T.: Prions prevent neuronal cell-line death. (Letter) Nature 400: 225-226, 1999.

Laplanche, J.-L.; El Hachimi, K. H.; Durieux, I.; Thuillet, P.; Defebvre, L.; Delasnerie-Laupretre, N.; Peoc'h, K.; Foncin, J.-F.; Destee, A.: Prominent psychiatric features and early onset in an inherited prion disease with a new insertional mutation in the prion protein gene. Brain 122: 2375-2386, 1999.

Laplanche, J. L.; Chatelain, J.; Thomas, S.; Launay, J. M.; Gaultier, C.; Derouesne, C. Uncommon phenotype for a codon 178 mutation of the human PrP gene. (Letter) Ann. Neurol. 31: 345, 1992.

Lee, H. S.; Sambuughin, N.; Cervenakova, L.; Chapman, J.; Pocchiari, M.; Litvak, S.; Qi, H. Y.; Budka, H.; del Ser, T.; Furukawa, H.; Brown, P.; Gajdusek, D. C.; Long, J. C.; Korczyn, A. D.; Goldfarb, L. G.: Ancestral origins and worldwide distribution of the PRNP 200K mutation causing familial Creutzfeldt-Jakob disease. Am. J. Hum. Genet. 64: 1063-1070, 1999.

Liao, Y.-C. J.; Lebo, J.; Lebo, R. V.; Clawson, G. A.; Smuckler, E. A.: Human prion protein cDNA: molecular cloning, chromosomal mapping, and biological implications. Science 233: 364-367, 1986.

Lindquist, S.: Mad cows meet Psi-chotic yeast: the expansion of the prion hypothesis. Cell 89: 495-498, 1997.

Little, B. W.; Brown, B. W.; Rodgers-Johnson, P.; Perl, D. P.; Gajdusek, D. C.: Familial myoclonic dementia masquerading as Creutzfeldt-Jakob disease. Ann. Neurol. 20: 231-239, 1986.

Lugaresi, E.; Medori, R.; Montagna, P.; Baruzzi, A.; Cortelli, P.; Lugaresi, A.; Tinuper, P. Zucconi, M.; Gambetti, P.: Fatal familial insomnia and dysautonomia with selective degeneration of thalamic nuclei. New Eng. J. Med. 315: 997-1003, 1986.

Lugaresi, E.; Montagna, P.; Baruzzi, A:.; Cortelli, P.; Tinuper, P.; Zucconi, M.; Gambetti, P. L.; Medori, R.: Insomnie familiale a evolution maligne: une nouvelle maladie thalamique. Rev. Neurol. 142: 791-792, 1986.

Mallucci, G. R.; Campbell, T. A.; Dickinson, A.; Beck, J.; Holt, M.; Plant, G.; de Pauw, K. W.; Hakin, R. N.; Clarke, C. E.; Howell, S.; Davies-Jones, G. A. B.; Lawden, M.; Smith, C. M. L.; Ince, P.; Ironside, J. W.; Bridges, L. R.; Dean, A.; Weeks, I.; Collinge, J.: Inherited prion disease with an alanine to valine mutation at codon 117 in the prion protein gene. Brain 122: 1823-1837, 1999.

Manetto, V.; Medori, R.; Cortelli, P.; Montagna, P.; Tinuper, P.; Baruzzi, A.; Rancurel, G.; Hauw, J.-J.; Vanderhaeghen, J.-J.; Mailleux, P.; Bugiani, O.; Tagliavini, F.; Bouras, C.; Rizzuto, N.; Lugaresi, E.; Gambetti, P.: Fatal familial insomnia: clinical and pathologic study of 5 new cases. Neurology 42: 312-319, 1992.

Manson, J. C.; Clarke, A. R.; McBride, P. A.; McConnell, I.; Hope, J.: PrP gene dosage determines the timing but not the final intensity or distribution of lesions in scrapie pathology. Neurodegeneration 3: 331-340, 1994.

Manuelidis, L.; Sklaviadis, T.; Manuelidis, E. E.: Evidence suggesting that PrP is not the infectious agent in Creutzfeldt-Jakob disease. EMBO J. 6: 341-347, 1987.

Mastrianni, J. A.; Curtis, M. T.; Oberholtzser, J. C.; Da Costa, M. M.; DeArmond, S.; Prusiner, S. B.; Garbern, J. Y.: Prion disease (PRP-A117V) presenting with ataxia instead of dementia. Neurology 45: 2042-2050, 1995.

Medori, R.: Personal Communication. New York, N.Y., May 17, 1990. Medori, R; Montagna, P.; Tritschler, H. J.; LeBlanc, A.; Cortelli, P.; Tinuper, P.; Lugaresi, E.; Gambetti, P.: Fatal familial insomnia: a second kindred with mutation of prion protein gene at codon 178. Neurology 42: 669-670, 1992.

Medori, R.; Tritschler, H.-J.: Prion protein gene analysis in thoursee kindreds with fatal familial insomnia (FFI): codon 178 mutation and codon 129 polymorphism. Am. J. Hum. Genet. 53: 822-827, 1993.

Medori, E.; Tritschler, H.-J.; LeBlanc, A.; Villare, F.; Manetto, V.; Chen, H. Y.; Xue, R.; Leal, S.; Montagna, P.; Cortelli, P.; Tinuper, P.; Avoni, P.; Mochi, M.; Baruzzi, A.; Hauw, J. J.; Ott, J.; Lugaresi, E.; Autilio-Gambetti, L.; Gambetti, P.: Fatal familial insomnia, a prion disease with a mutation at codon 178 of the prion protein gene. New Eng. J. Med. 326: 444-449, 1992.

Meggendorfer, F.: Klinische und genealogische Beobachtungen bei einem Fall von spastischer Pseudosklerose Jakobs. Z. Ges. Neurol. Psychiat. 128: 337-341, 1930. Meiner, Z.; Gabizon, R.; Prusiner, S. B.: Familial Creutzfeldt-Jakob disease: codon 200 prion disease in Libyan Jews. Medicine 76: 227-237, 1997.

Mestel, R.: Putting prions to the test. Science 273: 184-189, 1996.

Mitrova, E.; Lowenthal, A.; Appeal, B.: Familial Creutzfeldt-Jakob disease with temporal and spatial separation of affected members. Europ. J. Epidemiol. 6: 233-238, 1990.

Monari, L.; Chen, S. G.; Brown, P.; Parchi, P.; Petersen, R. B.; Mikol, J.; Gray, F.; Cortelli, P.; Montagna, P.; Ghetti, B.; Goldfarb, L. G.; Gajdusek, D. C.; Lugaresi, E.; Gambetti, P.; Autilio-Gambetti, L.: Fatal familial insomnia and familial Creutzfeldt-Jakob disease: different prion proteins determined by a DNA polymorphism. Proc. Nat. Acad. Sci. 91: 2839-2842, 1994.

Montrasio, F.; Frigg, R.; Glatzel, M.; Klein, M. A.; Mackay, F.; Aguzzi, A.; Weissmann, C.: Impaired prion replication in spleens of mice lacking functional follicular dendritic cells. Science 288: 1257-1259, 2000.

Mouillet-Richard, S.; Ermonval, M.; Chebassier, C.; Laplanche, J. L.; Lehmann, S.; Launay, J. M.; Kellermann, O.: Signal transduction thoursough prion protein. Science 289: 1925-1928, 2000.

Mouillet-Richard, S.; Teil, C.; Lenne, M.; Hugon, S.; Taleb, O.; Laplanche, J.-L.: Mutation at codon 210 (V2101) of the prion protein gene in a North African patient with Creutzfeldt-Jakob disease. J. Neurol. Sci. 168: 141-144, 1999.

Nieto, A.; Goldfarb, L. G.; Brown, P.; McCombie, W. R.; Trapp, S.; Asher, D. M.; Gajdusek, D. C.: Codon 178 mutation in ethnically diverse Creutzfeldt-Jakob disease families. (Letter) Lancet 337: 622-623, 1991.

Oesch, B.; Westaway, D.; Walchli, M.; McKinley, M. P.; Kent, S. B. H.; Aebersold, R.; Barry, R. A.; Tempst, P.; Teplow, D. B.; Hood, L. E.; Prusiner, S. B.; Weissmann, C.: A cellular gene encodes scrapie PrP 27-30 protein. Cell 40: 735-746, 1985.

Owen, F.; Poulter, M.; Collinge, J.; Crow, T. J.: A codon 129 polymorphism in the PRIP gene. Nucleic Acids Res. 18:3103, 1990.

Owen, F.; Poulter, M.; Collinge, J.; Crow, T. J.: Codon 129 changes in the prion protein gene in Caucasians. (Letter) Am. J. Hum. Genet. 46: 1215-1216, 1990.

Owen, F.; Poulter, M.; Collinge, J.; Leach, M.; Lofthouse, R.; Crow, T. J.; Harding, A. E. Molec. Brain Res. 13: 155-157, 1992.

Owen, F.; Poulter, M.; Lofthouse, R.; Collinge, J.; Crow, T. J.; Risby, D.; Baker, H. F.; Ridley, R. M.; Hsiao, K.; Prusiner, S. B.: Insertion in prion protein gene in familial Creutzfeldt-Jakob disease. (Letter) Lancet I: 51-52, 1989.

Owen, F.; Poulter, M.; Shah, T.; Collinge, J.; Lofthouse, R.; Baker, H.; Ridley, R.; McVey, J.; Crow, T. J.: An in-frame insertion in the prion protein gene in familial Creutzfeldt-Jakob disease. Molec. Brain Res. 7: 273-276, 1990.

Pablos-Mendez, A.; Netto, E. M.; Defendini, R.: Infectious prions or cytotoxic metabolites? Lancet341: 159-161, 1993.

Palmer, M. S.; Collinge, J.: Mutations and polymorphisms in the prion protein gene. Hum. Mutat 2: 168-173, 1993.

Palmer, M. S.; Dryden, A. J.; Hughes, J. T.; Collinge, J.: Homozygous prion protein genotype predisposes to sporadic Creutzfeldt-Jakob disease. Nature 352: 340-342, 1991. Note: Erratum: Nature 352: 547 only, 1991.

Perry, R. T.; Go, R. C. P.; Harrell, L. E.; Acton, R. T.: SSCP analysis and sequencing of the human prion protein gene (PRNP) detects two different 24 bp deletions in an a typical Alzheimer's disease family. Am. J. Med. Genet. 60: 12-18, 1995.

Pocchiari, M.; Salvatore, M.; Cutruzzola, F.; Genuardi, M.; Allcatelli, C. T.; Masullo, C.; Macchi, G.; Alema, G.; Galgani, S.; Xi, Y. G.; Petraroli, R.; Silvestrini, M. C.; Brunori, M.: A new point mutation of the prion protein gene in Creutzfeldt-Jakob disease. Ann. Neurol. 34: 802-807, 1993.

Poulter, M.; Baker, H. F.; Frith, C. D.; Leach, M.; Lofthouse, R.; Ridley, R. M.; Shah, T.; Owen, F.; Collinge, J.; Brown, J.; Hardy, J.; Mullan, M. J.; Harding, A. E.; Bennett, C.; Doshi, R.; Crow, T. J.: Inherited prion disease with 144 base pair gene insertion. 1. Genealogical and molecular studies. Brain 115: 675-685, 1992.

Prusiner, S. B.: Prions causing degenerative neurological diseases. Annu. Rev. Med. 38: 381-398, 1987.

Prusiner, S. B.: Molecular biology of prion diseases. Science 252: 1515-1522, 1991.

Prusiner, S. B.: Biology and genetics of prion diseases. Ann. Rev. Microbiol. 48: 655-686, 1994.

Prusiner, S. B.: Novel proteinaceous infectious particles cause scrapie. Science 216: 136-144, 1982.

Prusiner, S. B.: Molecular biology and genetics of prion diseases. Cold Spring Harbor Symp. Quant. Biol. 61: 473-493, 1996.

Puckett, C.; Concannon, P.; Casey, C.; Hood, L.: Genomic structure of the human prion protein gene. Am. J. Hum. Genet. 49: 320-329,1991.

Reder, A. T.; Mednick, A. S.; Brown, P.; Spire, J. P.; Cauter, V.; Wollmann, R. L.; Cervenakova, L.; Goldfarb, L. G.; Garay, A.; Ovsiew, F.; Gajdusek, D. C.; Roos, R. P. Clinical and genetic studies of fatal familial insomnia. Neurology 45: 1068-1075, 1995.

Riek, R.; Wider, G.; Billeter, M.; Hornemann, S.; Glockshuber, R.; Wuthoursich, K.: Prion protein NMR structure and familial human spongiform encephalopathies. Proc.Nat. Acad. Sci. 95: 11667-11672, 1998.

Rivera, H.; Zuffardi, O.; Maraschio, P.; Caiulo, A.; Anichini, C.; Scarinci, R.; Vivarelli, R.: Alternate centromere inactivation in a pseudodicentric (15;20)(pter;pter) associated with a progressive neurological disorder. J. Med. Genet. 26: 626-630, 1989.

Robakis, N. K; Devine-Gage, E. A.; Jenkins, E. C.; Kascsak, R. J.; Brown, W. T.; Krawczun, M. S.; Silverman, W. P.: Localization of a human gene homologous to the PrP gene on the p arm of chromosome 20 and detection of PrP-related antigens in normal human brain. Biochem. Biophys. Res. Commun. 140: 758-765, 1986.

Sailer, A.; Bueler, H.; Fischer, M.; Aguzzi, A.; Weissmann, C.: No propagation of prions in mice devoid of PrP. Cell 77: 967-968, 1994.

Sakaguchi, S.; Katamine, S.; Nishida, N.; Moriuchi, R.; Shigamatsu, K.; Sugimoto, T.; Nakatani, A.; Kataoka, Y.; Houtani, T.; Shirabe, S.; Okada, H.; Hasegawa, S.; Miyamoto, T.; Noda, T.: Loss of cerebellar Purkinje cells in aged mice homozygous for a disrupted PrP gene. Nature 380: 528-531, 1996.

Samaia, H. B.; Mari, J. J.; Vallada, H. P.; Moura, R. P.; Simpson, A. J. G.; Brentani, R. R.: A prion-linked psychiatric disorder. Nature 390: 241 only, 1997.

Schellenberg, G. D.; Anderson, L.; O'dahl, S.; Wisjman, E. M.; Sadovnick, A. D.; Ball, M. J.; Larson, E. B.; Kukull, W. A.; Martin, G. M.; Roses, A. D.; Bird, T. D.: APP-717, APP-693, and PRIP gene mutations are rare in Alzheimer disease. Am. J. Hum. Genet. 49: 511-517, 1991.

Schnittger, S.; Gopal Rao, V. V. N.; Deutsch, U.; Gruss, P.; Balling, R.; Hansmann, I.: PAX1, a member of the paired box-containing class of developmental control genes, is mapped to human chromosome 20p11.2 by in situ hybridization (ISH and FISH). Genomics 14: 740-744, 1992.

Scott, M.; Foster, D.; Mirenda, C.; Serban, D.; Coufal, F.; Walchli, M.; Torchia, M.; Groth, D.; Carlson, G.; DeArmond, S. J.; Westaway, D.; Prusiner, S. B.: Transgenic mice expressing hamster prion protein produce species-specific scrapie infectivity and amyloid plaques. Cell 59: 847-857, 1989.

Shibuya, S.; Higuchi, J.; Shin, R.-W.; Tateishi, J.; Kitamoto, T.: Protective prion protein polymorphisms against sporadic Creutzfeldt-Jakob disease. (Letter) Lancet 351: 419 only, 1998.

Shmerling, D.; Hegyi, I.; Fischer, M.; Blattler, T.; Brandner, S.; Gotz, J.; Rulicke, T.; Flechsig, E.; Cozzio, A.; von Mering, C.; Hangartner, C.; Aguzzi, A.; Weissmann, C.: Expression of amino-terminally truncated PrP in the mouse leading to ataxia and specific cerebellar lesions. Cell 93: 203-214, 1998.

Simon, E. S.; Kahana, E.; Chapman, J.; Treves, T. A.; Gabizon, R.; Rosenmann, H.; Zilber, N.; Korczyn, A. D.: Creutzfeldt-Jakob disease profile in patients homozygous for the PRNP E200K mutation. Ann. Neurol. 47: 257-260, 2000.

Sparkes, R. S.; Simon, M.; Cohn, V. H.; Fournier, R. E. K.; Lem, J.; Klisak, I.; Heinzmann, C.; Blatt, C.; Lucero, M.; Mohandas, T.; DeArmond, S. J.; Westaway, D.; Prusiner, S. B.; Weiner, L. P.: Assignment of the human and mouse prion protein genes to homologous chromosomes. Proc. Nat. Acad. Sci. 83: 7358-7362, 1986.

Speer, M. C.; Goldgaber, D.; Goldfarb, L. G.; Roses, A. D.; Pericak-Vance, M. A.: Support of linkage of Gerstmann-Straussler-Scheinker syndrome to the prion protein gene on chromosome 20 p12-pter. Genomics 9: 366-368, 1991.

Supattapone, S.; Bosque, P.; Muramoto, T.; Wille, H.; Aagaard, C.; Peretz, D.; Nguyen, H.-O. B.; Heinrich, C.; Torchia, M.; Safar, J.; Cohen, F. E.; DeArmond, S. J.; Prusiner, S. B.; Scott, M.: Prion protein of 106 residues creates an artificial transmission barrier for prion replication in transgenic mice. Cell 96: 869-878, 1999.

Tagliavini, F.; Prelli, F.; Ghiso, J.; Bugiani, O.; Serban, D.; Prusiner, S. B.; Farlow, M. R.; Ghetti, B.; Frangione, B.: Amyloid protein of Gerstmann-Straussler-Scheinker disease (Indiana kindred) is an 11 kd fragment of prion protein with an N-terminal glycine at codon 58. EMBO J. 10: 513-519, 1991.

Tagliavini, F.; Prelli, F.; Porro, M.; Rossi, G.; Giaccone, G.; Farlow, M. R.; Dlouhy, S. R.; Ghetti, B.; Bugiani, O.; Frangione, B.: Amyloid fibrils in Gerstmann-Straussler-Scheinker disease (Indiana and Swedish kindreds) express only PrP peptides encoded by the mutant allele. Cell 79: 695-703, 1994.

Tateishi, J.; Brown, P.; Kitamoto, T.; Hoque, Z. M.; Roos, R.; Wollman, R.; Cervenakova, L.; Gajdusek, D. C.: First experimental transmission of fatal familial insomnia. Nature 376: 434-435, 1995.

Telling, G. C.; Parchi, P.; DeArmond, S. J.; Cortelli, P.; Montagna, P.; Gabizon, R.; Mastrianni, J.; Lugaresi, E.; Gambetti, P.; Prusiner, S. B.: Evidence for the conformation of the pathologic isoform of the prion protein enciphering and propagating prion diversity. Science 274: 2079-2082, 1996.

Telling, G. C.; Scott, M.; Mastrianni, J.; Gabizon, R.; Torchia, M.; Cohen, F. E.; DeArmond, S. J.; Prusiner, S. B.: Prion propagation in mice expressing human and chimeric PrP transgenes implicates the interaction of cellular PrP with another protein. Cell 83: 79-90, 1995.

Ter-Avanesyan, M. D.; Dagkesamanskaya, A. R.; Kushnirov, V. V.; Smirnov, V. N.: The SUP35 omnipotent suppressor gene is involved in the maintenance of the non-mendelian determinant [psi+] in the yeast Saccharomyces cerevisiae. Genetics 137: 671-676, 1994.

Tobler, I.; Gaus, S. E.; Deboer, T.; Ackermann, P.; Fischer, M.; Rullcke, T.; Moser, M.; Oesch, B.; McBride, P. A.; Manson, J. C.: Altered circadian activity rhythms and sleep in mice devoid of prion protein. Nature 380: 639-642, 1996.

Westaway, D.; DeArmond, S. J.; Cayetano-Canlas, J.; Groth, D.; Foster, D.; Yang, S.-L.; Torchia, M.; Carlson, G. A.; Prusiner, S. B.: Degeneration of skeletal muscle, peripheral nerves, and the central nervous system in transgenic mice overexpressing wild-type prion proteins. Cell 76: 117-129, 1994.

Whittington, M. A.; Sidle, K C. L.; Gowland, I.; Meads, J.; Hill, A. F.; Palmer, M. S, Jefferys, J. G. R.; Collinge, J.: Rescue of neurophysiological phenotype seen in PrP null mice by transgene encoding human prion protein. Nature Genet. 9: 197-201, 1995.

Wickner, R. B.: [URE3] as an altered URE2 protein: evidence for a prior analog in Saccharomyces cerevisiae. Science 264: 566-569, 1994.

Windl, O.; Giese, A.; Schulz-Schaeffer, W.; Zerr, I.; Skworc, K.; Arendt, S.; Oberdieck, C.; Bodemer, M.; Poser, S.; Kretzschmar, H. A.: Molecular genetics of human prion diseases in Germany. Hum. Genet. 105: 244-252, 1999.

Yamada, M.; Itoh, Y.; Fujigasaki, H.; Naruse, S.; Kaneko, K.; Kitamoto, T.; Tateishi, J.; Otomo, E.; Hayakawa, M.; Tanaka, J.; Matsushita, M.; Miyatake, T.: A missense mutation at codon 105 with codon 129 polymorphism of the prion protein gene in a new variant of Gerstmann-Straussler-Scheinker disease. Neurology 43: 2723-2724, 1993.

1. A method for detecting the presence of prions in a tissue/organ; said method comprising the steps of:
(a) contacting the tissue/organ with a device, wherein said device is capable of binding prions;
(b) removing said device from contact with said tissue/organ; and
(c) determining if said device is binding prions.

2. A non-invasive method for detecting the presence of prions in a tissue/organ; said method comprising the steps of:
(a) contacting the tissue/organ with a device, wherein said device is capable of binding prions;
(b) removing said device from contact with said tissue/organ; and
(c) determining if said device is binding prions.

3. A method according to claims 1 or claim 2 wherein the device is capable of preserving prions against degradation.

4. A method according to any of claims 1 to 3 wherein the tissue/organ is a mammalian tissue/organ.

5. A method according to claim 4 wherein the tissue/organ is a livestock or a human tissue/organ.

6. A method according to any of claims 1 to 5 wherein the tissue/organ is an tissue/organ in which prions accumulate.

7. A method according to claim 6 wherein the tissue/organ is selected from brain, spleen, lymph node or tonsil.

8. A method according to any preceding claim wherein the device comprises metal.

9. A method according to claim 8 wherein the metal comprises one or more metal(s) selected from the group consisting of steel, stainless steel, silver, gold or combinations thereof.

10. A method according to claim 8 or claim 9 wherein the device(s) comprise one or more wires.

11. A method for determining if a device is binding prions comprising the steps of:
contacting one or more test animal(s) with a device;
incubating the test animal(s);
monitoring the test animal(s) for adverse effects or death; and optionally performing a biopsy on any test animal(s) that display adverse effects or death for evidence of prions.

12. A method according to claim 11 wherein said device is contacted with one or more test animals for 1 hour or more per test animal.

13. A method according to claim 12 wherein said device is contacted with one or more test animals for 5 hours or more per test animal.

14. A method according to claim 13 wherein said device is contacted with one or more test animals for more than 5 hours per test animal.

15. A method according to any one of claims 11 to 14 wherein the test animal(s) are mammals.

16. A method according to claim 15 wherein the test animal(s) are mice.

17. A method according to claim 16 wherein the test animal(s) are transgenic mice.

18. A method according to claim 17 wherein the transgenic mice comprise one or more PrP transgene(s).

19. A method according to claim 18 wherein the PrP transgene(s) encode a mammalian PrP.

20. A method according to claim 19 wherein the PrP transgene(s) encode a livestock or a human PrP.

21. A method for determining if a device is binding prions comprising the steps of:
(a) contacting a cell line with a device;
(b) incubating the cell line; and
(c) determining if the cell line contain prions.

22. A method according to claim 21 wherein it is determined if the cell line(s) contain prions using a protein assay, an immunoassay, Western blotting or cell blotting.

23. A method for determining if a device is binding prions by detecting said prions directly on the surface of said device.

24. A method according to claim 23 wherein it is determined if prions are bound to the surface of said device using a protein assay, an immunoassay or Western blotting.

25. A method according to any of claims 1 to 24 wherein the device is contacted with the tissue/organ for 120 minutes or less.

26. A method according to any of claims 1 to 24 wherein the device is contacted with the tissue/organ for 30 minutes or less.

27. A method according to any of claims 1 to 24 wherein the device is contacted with the tissue/organ for 5 minutes or less.

28. A device capable of binding prions, wherein said device comprises metal.

29. A device according to claim 28 wherein the device comprises any one or more metal(s) selected from the group consisting of steel, stainless steel, silver, gold or combinations thereof.

30. A device according to claim 28 or claim 29 wherein said device comprises one or more wires.

31. A device as defined in any one of claims 28 to 30, wherein prions are preserved when bound to said device.
